(19) United States
(12) Patent Application Publication
(10) Pub. No.: US 2024/0049668 A1
(43) Pub. Date: Feb. 15, 2024

(54) TOMATO PLANTS RESISTANT TO TOBRFV, TMV, TOMV AND TOMMV AND CORRESPONDING RESISTANCE GENES

(71) Applicant: VILMORIN & CIE, Paris (FR)

(72) Inventor: John LINDBO, Davis, CA (US)

(73) Assignee: VILMORIN & CIE, Paris (FR)

(21) Appl. No.: 18/255,916

(22) PCT Filed: Dec. 3, 2021
(86) PCT No.: PCT/EP2021/084289

§ 371 Date: Jun. 5, 2023

(30) Foreign Application Priority Data
Dec. 3, 2020 (EP) 20306496.9
Aug. 2, 2021 (EP) 21306078.3

(51) Int. Cl. A01H 1/00; C07K 14/415; C12N 15/82

(52) U.S. Cl. CPC A01H 1/126; C07K 14/415; C12N 15/8283

(57) ABSTRACT

A variants of the TM-2-2 protein, conferring recognition of the Movement Protein (MP) of the Tomato Brown Rugose Fruit Virus (ToBRFV), and wherein said variant comprises a tyrosine (Y), a phenylalanine (F) or a tryptophan (W) at the position corresponding to tyrosine 767 of the TM-2-2 protein and at least one of the following mutations: C848R, N822C, N822F, N822M, N822Y, N822W, N825H, N825K and N825T with respect to the TM-2-2 protein, potentially in combination with a F655L mutation. The present invention also relates to genetic sequences encoding such a variant protein, preferably to a mutated Tm-2-2 gene, and to plants, especially Solanum lycopersicum plants comprising in their genome the mutated gene conferring resistance to ToBRFV. The invention is also directed to parts of these plants, as well as progeny, and to the use of these sequences for providing ToBRFV resistance.

The present invention relates to resistance in plants of Solanum lycopersicum, also known as Lycopersicum esculentum, to tobamoviruses, especially to the Tomato Brown Rugose Fruit virus (ToBRFV, previously abbreviated TBRFV), and preferably also to Tobacco Mosaic Virus (TMV), Tomato Mosaic Virus (ToMV) and/or Tomato Mottle Mosaic Virus (ToMMV). More specifically, the present invention relates to tomato plants and fruits comprising a resistance gene that leads to resistance to at least ToBRFV and preferably to at least one additional tobamovirus. According to the invention, the resistance gene conferring resistance to these tobamoviruses is a variant of the Tm-2 and Tm-2-2 gene alleles. The resistance gene can be present homozygously or heterozygously in the genome of a S. lycopersicum plant. The invention further relates to this resistance gene, parts thereof, to encoded polypeptides and proteins and to the use of these sequences and proteins to obtain resistant plants. The invention also relates to the seeds and progeny of such plants, to propagation material for obtaining such plants, and to different uses of these plants.

### BACKGROUND OF THE INVENTION

All cultivated and commercial forms of tomato belong to a species most frequently referred to as Lycopersicon esculentum Miller. Lycopersicon is a relatively small genus within the extremely large and diverse family Solanaceae which is considered to consist of around 90 genera, including pepper, tobacco and eggplant. The genus Lycopersicon has been divided into two subgenera, the esculentum complex which contains those species that can easily be crossed with the commercial tomato and the peruvianum complex which contains those species which are crossed with considerable difficulty. Due to its value as a crop, L. esculentum Miller has become widely disseminated all over the world.

Tomato is grown for its fruit, widely used as a fresh market or processed product. As a crop, tomato is grown commercially wherever environmental conditions permit the production of an economically viable yield. The majority of fresh market tomatoes are harvested by hand at vine ripe and mature green stage of ripeness. Fresh market tomatoes are available year round. Processing tomato are mostly mechanically harvested and used in many forms, as canned tomatoes, tomato juice, tomato sauce, puree, paste or even catsup.

Tomato is a normally simple diploid species with twelve pairs of differentiated chromosomes. However, polyploidy tomato is also part of the present invention. The cultivated tomato is self-fertile and almost exclusively self-pollinating. The tomato flowers are hermaphrodites. Commercial cultivars were initially open pollinated. As hybrid vigor has been identified in tomatoes, hybrids are replacing the open pollinated varieties by gaining more and more popularity amongst farmers with better yield and uniformity of plant characteristics. Due to its wide dissemination and high value, tomato has been intensively bred. This explains why such a wide array of tomato is now available. The shape may range from small to large, and there are cherry, plum, pear, blocky, round, and beefsteak types.

Tomatoes may be grouped by the amount of time it takes for the plants to mature fruit for harvest and, in general the cultivars are considered to be early, midseason or late-maturing. Tomatoes can also be grouped by the plant's growth habit; determinate, semi-determinate or indeterminate. Determinate plants tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties. More recent developments in tomato breeding have led to a wider array of fruit color. In addition to the standard red ripe color, tomatoes can be creamy white, lime green, pink, yellow, golden, orange or purple. Hybrid commercial tomato seed can be produced by hand pollination. Pollen of the male parent is harvested and manually applied to the stigmatic surface of the female inbred. Prior to and after hand pollination, flowers are covered so that insects do not bring foreign pollen and create a mix or impurity. Flowers are tagged to identify pollinated fruit from which seed will be harvested.

A variety of pathogens affect the productivity of tomato plants, including virus, fungi, bacteria, nematodes and insects. Tomatoes are inter alia susceptible to many viruses and virus resistance is therefore of major agricultural importance.

Tobamoviruses are among the most important plant viruses causing severe damages in agriculture, especially to vegetable and ornamental crops around the world. Tobamoviruses are easily transmitted by mechanical means, as well as through seed transmission. Tobamoviruses are generally characterized by a rod-shaped particle of about 300 nm encapsidating a single stranded, positive RNA genome encoding four proteins. In tomatoes, tobacco mosaic virus (TMV) and tomato mosaic virus (ToMV) are feared by growers worldwide as they can severely damage crop production, for example through irregular ripening (fruits having yellowish patches on the surface and brownish spots beneath the surface). Several genes have however been identified by plants breeders over the years and TMV and/or ToMV resistant tomato varieties are nowadays available.

Another tobamovirus, namely the Tomato mottle mosaic virus (ToMMV), has been recently described infecting tomato plants in several countries worldwide, reducing annual yield and quality of tomato production.

Tobamoviruses belong to the a-like supergroup of viruses. They have a proteinaceous rod, made up of copies of the coat protein (CP), which envelops the linear RNA(+) genome. After infection of the plant cell, the RNA genome is uncoated and transcribed, producing the RNA-dependent RNA polymerase (RdRP), the movement protein (MP) and the coat protein (CP). Infection of neighboring cells as well as long-distance transport of the virus are dependent on the movement protein (MP). Resistance against pathogens like tobamoviruses requires the presence of resistance (R) genes, whose polypeptide products, R proteins, recognize products of the tobamoviruses and, subsequently, are able to trigger a defense response, generally hypersensitive response.

For the last decades, all modern indeterminate tomato varieties and many of the determinate tomato varieties indeed contain the Tm-2 gene or preferably the Tm-2² (also known as Tm-2-2) allele of this gene, as resistance gene. These genes, introgressed from S. peruvianum, indeed give them immunity to almost all known races of Tobamoviruses which affected commercial tomatoes (ToMV and TMV) before 2014. The resistance gene Tm-2-2 appears to also mostly confer resistance to ToMMV (Nagai et al, 2019; Sui et al, 2017).

The Tm-2 (SEQ ID No:2) and the Tm-2-2 (SEQ ID No:3) resistance genes are considered to be allelic and share the movement protein (MP) of ToMV as the matching avirulence (Avr) protein. The TM-2 and TM-2-2 proteins (SEQ ID No:7 and 8 respectively) have the characteristics of the nucleotide binding site/leucine-rich repeat type of R proteins (NBS-LRR proteins, also known as NLR proteins) and differ considerably from the polypeptide (SEQ ID No:6) encoded by the tm-2 allele (SEQ ID No:1) isolated from susceptible L. esculentum lines (Lanfermeijer et al. 2003). Tm-2 and Tm-2-2 mediated resistance requires that these NLR proteins recognize a tobamovirus movement protein (MP) (Calder and Palukaitis 1992; Meshi et al. 1989; Weber and Pfitzner 1998; Weber et al. 1993).

The differences between the products of the tm2 and Tm-2 genes are concentrated in the leucine-rich repeat (LRR) domain, as well as the difference between the Tm-2 and Tm-2-2 genes (Lanfermeijer et al. 2005).

During 2014-2015, a severe outbreak of virus affected tomato productions areas in the middle east, such as in Jordan and in Israel. Most of the tomato varieties affected were considered TMV and/or ToMV resistant, but were still severely affected and showed typical TMV/ToMV like symptoms: while the foliar ones were quite similar to the TMV/ToMV symptoms, the fruit symptoms were much more frequent and severe than the usual symptoms from such viruses with fruits lesions and deformations. The fruit quality was very poor and rather unmarketable. Salem et al, 2015 sequenced this new Tobamovirus species, and proposed to name this Jordanian virus: Tomato Brown Rugose Fruit virus (previously TBRFV and now ToBRFV). The comparison to other Tobamoviruses sequences showed that it is indeed a Tobamovirus, but not TMV or ToMV. The resistance to TMV and/or ToMV does not confer resistance to this new virus ToBRFV; i.e plants comprising either the Tm-2 or the Tm-2-2 resistance gene are still susceptible to ToBRFV.

Luria et al, 2017 have concomitantly isolated and sequenced the complete genome of the Israeli tobamovirus infecting tomato in Israel, they have shown a very high sequence identity between the Israeli and the Jordanian viruses (more than 99% sequence identity) and have concluded to two different isolates of tomato brown rugose fruit virus.

Recently, the virus was identified in Europe, especially in Sicily, Germany, the Netherlands and France, and in Mexico, and therefore now it is considered as a major global threat to tomato crop. Identification of a resistance gene against this new tobamovirus has thus become important and urgent for tomato breeders.

Identification of tomato plants which display resistance to ToBRFV and localization and identification of genetic determinants, also referred to hereafter as QTLs (Quantitative Trait Locus) that lead to resistance to the Tomato Brown Rugose Fruit virus have recently been described in WO2018/219941. Two QTLs, namely QTL1 and QTL2, on chromosome 6 and 9 respectively, confer independently or in combination an improved tolerance or resistance in the fruits of a tomato plant infected or likely to be infected by the ToBRFV, when present homozygously into a S. lycopersicum background. A third QTL, QTL3, on chromosome 11, confers an improved tolerance or resistance in the leaves of a tomato plant infected or likely to be infected by the ToBRFV, when present homozygously.

Whereas these QTLs, either alone or in combination, provide tolerance or resistance to ToBRFV, this tolerance/resistance appears quantitative and polygenic, and plants are not free of virus. Moreover, these QTLs are described as providing resistance when present homozygously. Insofar as QTL2, on chromosome 9, is present at the same locus as the Tm-2-2 gene, in a region which is generally transmitted “en bloc” without recombination, such a QTL on chromosome 9 is therefore likely to be difficult to combine with the Tm-2-2 gene which remains mandatory for commercial plants. WO2020/148021 recently described a resistance gene, on chromosome 8, conferring resistance to ToBRFV; this gene codes for a NBS-LRR protein (Nucleotide-binding site Leucine-rich repeat). The resistance gene disclosed in this document is thus to be combined with the Tm-2-2 resistance gene in order to provide resistance simultaneously to ToBRFV, TMV and ToMV.

Since Tobamovirus particles are very stable and highly infectious, their prevention is generally very difficult. Therefore, one of the most effective ways of combating tobamoviruses infections is the introduction of genetic resistance gene. There is thus an urgent need to identify improved resistance genes against several tobamoviruses simultaneously, including this new Tobamovirus ToBRFV, failing that would result in entire regions in which tomato crop could not be produced anymore. Plant NLR proteins have been extensively reviewed. (Baggs et al. 2017; Kapos et al. 2019). NLRs are proteins that upon recognition of an ‘effector protein’ trigger a resistance response in plants. NLRs have been grouped into two major subclasses according to their N-terminal domain. The two major subclasses are CNLs and TNLs for proteins that contain either a coiled coil (C) or a toll/interleukin 1 receptor (TIR) domain, respectively at their N-terminus. In addition to the N-terminal domain NLRs also have a nucleotide binding (NB) domain and a Leucine Rich Repeat (LRR) domain. Each of the common domains (NB, LRR, CC and TIR) are proposed to play a role in activation of the NLR Protein (Wang et al. 2020). The LRR domain in particular is usually associated with effector protein recognition. As a result, allelic diversity in the LRR domain is often associated with specificity of effector protein binding and LRR domain diversity appears to be under positive selection (Mondragon-Palomino et al. 2002). The LRR domain can also act as an auto-inhibitory domain preventing auto-activation and downstream signaling. The NB domain binds ATP and can switch between an active or inactive form. The CC and TIR domains are generally thought to be involved in signaling. There are also reports where transferring part of a LRR domain between two NLR proteins could confer new specificity to an NLR protein (Slootweg et al. 2017).

In the case of the NLR proteins TM2/TM-2-2 it has been demonstrated that a single amino acid (AA) change in the LRR domain is responsible for expanding the diversity of tobamovirus Movement proteins (MP) recognized by the protein (Kobayashi et al. 2011).

The present inventors have unexpectedly found that, contrary to the teaching of the prior art, resistance to TMV, ToMV and ToBRFV can be conferred by a single resistance protein, encoded by a single resistant gene, without requiring the combination of different resistance genes and proteins. They have moreover found that such a protein conferring resistance to ToBRFV can be obtained by modifying the recognition domain, namely the LRR domain, of a NLR protein conferring resistance to TMV and ToMV, namely by modifying the LRR domain of a TM-2-2 protein.

### SUMMARY

The present invention is directed to a variant LRR domain, deriving from the LRR domain of the TM-2-2 protein, such that a NBS-LRR protein comprising said mutant LRR domain recognizes and/or binds the Movement Proteins of several different tobamoviruses, including at least ToBRFV, and preferably also TMV, ToMV and/or ToMMV, most preferably TMV, ToMV and ToMMV.

The invention is also directed to polypeptides comprising such a mutant LRR domain, as well as nucleotide sequences encoding said domains and polypeptides.

The invention also concerns a Resistance gene, encoding a mutant or allelic variant of the TM-2-2 protein (SEQ ID No:8), conferring to tomatoes resistance against several tobamoviruses, including at least against ToBRFV.

The newly discovered resistance protein or LRR domain confers resistance against ToBRFV, preferably in addition to resistance against ToMV and TMV, thanks to at least one substitution in the LRR domain of the TM-2-2 protein, at position 822, 825 or 848, namely the substitution of the asparagine (N) 822 by a cysteine (C), a phenylalanine (F), a methionine (M), a tyrosine (Y) or a tryptophan (VW), the substitution of the serine (S) 825 by a histidine (H), a lysine (K) or a threonine (T), the substitution of the cysteine (C) 848 by an arginine (R), or a combination of these substitutions. Additional substitutions, and preferably the substitution of the phenylalanine (F) at position 655 by a leucine (L), may improve the resistance conferred by the substitution at position 848, 822 and/or 825, especially substitution at position 848.

The present invention also provides plants, especially S. lycopersicum plants that display resistance to ToBRFV, including commercial plants, lines and hybrids, as well as methods that produce or identify plants, especially S. lycopersicum plants or populations (germplasm) that display resistance to ToBRFV. The present invention also discloses molecular genetic markers, linked to the newly discovered resistance gene. Plants obtained through the methods and uses of such molecular markers are also provided.

The invention also provides several methods, including methods for identifying ToBRFV resistant plants, methods for improving the yield of tomato production in an environment infested by different tobamoviruses including ToBRFV, methods for protecting a tomato field from tobamoviruses infestation including ToBRFV and methods for identifying, detecting and/or selecting mutants of the Tm-2-2 or Tm-2 gene conferring resistance against at least ToBRFV.

### Definitions

The term “Resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure. Two levels of resistance are defined:

High Resistance: plants that highly restrict the growth and/or development of the specified pest and/or the damage it causes under normal pest pressure when compared to susceptible plants. These plants may, however, exhibit some symptoms or damage under heavy pest pressure.

Intermediate Resistance: plants that highly restrict the growth and/or development of the specified pest and/or the damage it causes but may exhibit a greater range of symptoms or damage compared to high resistance plants. Intermediate resistant plants will still show less severe symptoms or damage than susceptible plants when grown under similar environmental conditions and/or pest pressure.

The term “Tolerance” is normally used to describe the ability of a plant to endure abiotic stresses without serious consequences for growth, appearance and yield.

In the literature and patents, this term is however also used to indicate a phenotype of a plant wherein at least some of the disease-symptoms remain absent upon exposure of said plant to an infective dose of virus, whereby the presence of a systemic or local infection, virus multiplication, at least the presence of viral genomic sequences in cells of said plant and/or genomic integration thereof can be established, at least under some culture conditions. Tolerant plants are therefore resistant for symptom expression but symptomless carriers of the virus. Sometimes, viral sequences may be present or even multiply in plants without causing disease symptoms. It is to be understood that a tolerant plant, although it is infected by the virus, is generally able to restrict at least moderately the growth and development of the virus. For this reason, tolerant plants according to this definition are best characterized by Intermediate Resistant plants.

Symptoms on leaves of ToBRFV infection generally include mosaic, distortion of the leaflets and in many cases also shoestrings like symptoms. Symptoms on fruits of ToBRFV infection generally include typical yellow lesions (discoloration) and deformation of the fruits. In many cases there are also “chocolate spots” on the fruits.

Susceptibility: The inability of a plant to restrict the growth and development of a specified pest or pathogen; a susceptible plant displays the detrimental symptoms linked to the virus infection, namely the foliar damages and fruit damages in case of ToBRFV infection.

A S. lycopersicum plant susceptible to Tomato Brown Rugose Fruit virus, is for example the commercially available variety Candela as mentioned in the 2015 Salem et al. publication.

As used herein, the term “offspring” or “progeny” refers to any plant resulting as progeny from a vegetative or sexual reproduction from one or more parent plants or descendants thereof. For instance, an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (a true-breeding parent is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “genotype” refers to the genetic makeup of an individual cell, cell culture, tissue, organism (e.g., a plant), or group of organisms.

As used herein, the term “grafting” is the operation by which a rootstock is grafted with a scion. The primary motive for grafting is to avoid damages by soil-born pest and pathogens when genetic or chemical approaches for disease management are not available. Grafting a susceptible scion onto a resistant rootstock can provide a resistant cultivar without the need to breed the resistance into the cultivar. In addition, grafting may enhance tolerance to abiotic stress, increase yield and result in more efficient water and nutrient uses.

As used herein, the term “heterozygote” refers to a diploid or polyploid individual cell or plant having different alleles (forms of a given gene, genetic determinant or sequences) present at least at one locus.

As used herein, the term “heterozygous” refers to the presence of different alleles (forms of a given gene, genetic determinant or sequences) at a particular locus.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “locus” (plural: “loci”) refers to any site that has been defined genetically, this can be a single position (nucleotide) or a chromosomal region. A locus may be a gene, a genetic determinant, a part of a gene, or a DNA sequence, and may be occupied by different sequences. A locus may also be defined by a SNP (Single Nucleotide Polymorphism), by several SNPs, or by two flanking SNPs.

As used herein, the term “rootstock” is the lower part of a plant capable of receiving a scion in a grafting process.

As used herein, the term “scion” is the higher part of a plant capable of being grafted onto a rootstock in a grafting process.

The invention encompasses plants of different ploidy levels, essentially diploid plants, but also triploid plants, tetraploid plants, etc.

### DETAILED DESCRIPTION OF THE INVENTION

The present inventors have identified variants of the LRR domain of the TM-2-2 protein, such that a NBS-LRR protein comprising said variants of the LRR domain recognizes and/or binds the Movement Proteins (MP) of several tobamoviruses, including at least ToBRFV (Tomato Brown Rugose Fruit Virus) and preferably also MP of TMV (Tobacco Mosaic Virus) and/or ToMV (Tomato Mosaic Virus), and preferably also ToMMV (Tomato Mottle Mosaic Virus).

The present invention is thus directed to variants of the Leucine-Rich Repeat of the TM-2-2 protein, namely variants of SEQ ID No:11, wherein said variants have at least 90% sequence identity with SEQ ID No:11 and said variants confer, if incorporated into a NBS-LRR protein, the capacity to recognize and/or bind at least the Movement Protein of the ToBRFV (SEQ ID No:15); and preferably also the MP of the ToMV (SEQ ID No:14) and/or of the TMV (SEQ ID No:13), and still preferably also the MP of ToMMV (SEQ ID No:16). By way of contrast, the LRR domain of TM-2-2, i.e. SEQ ID No:11, confers the capacity to bind MP of ToMV and TMV, but does not confer significant binding to or recognition of MP of ToBRFV. Variants of the LRR domain of TM-2-2 of the invention are interchangeably referred to as LRR variants or mutants, or LRR domain variants or mutants, or LRR variant domains of the invention.

Substitution of the LRR domain of TM-2-2, i.e. SEQ ID No:11, by a LRR variant of the invention, confers to the thus obtained protein recognition of the ToBRFV MP, i.e. results in a protein recognizing ToBRFV MP.

A NBS-LRR protein is said to recognize the ToBRFV MP if said protein binds, directly or indirectly, the ToBRFV MP; the direct or indirect binding may be at the LRR domain level, or may involve the whole protein. Such a recognition can be tested by the assay disclosed in the examples.

The inventors have moreover demonstrated that the variation conferring recognition of ToBRFV MP lies in the substitution of at least one of the amino acids at position 822, 825 and 848 of the TM-2-2 protein (SEQ ID No:8), and more specifically in at least one of the substitutions consisting in:
the substitution of the cysteine (C) at position 848 of the TM-2-2 protein (SEQ ID No:8), corresponding to position 372 of SEQ ID No:11 (LRR domain of TM-2-2, corresponding to amino acids 477 to 861 of TM-2-2 protein), by an arginine (R),
the substitution of the asparagine (N) at position 822 of the TM-2-2 protein (SEQ ID No:8), corresponding to position 346 of SEQ ID No:11 (LRR domain of TM-2-2), by a cysteine (C), a phenylalanine (F), a methionine (M), a tyrosine (Y) or a tryptophan (VW), and
the substitution of the serine (S) at position 825 of the TM-2-2 protein (SEQ ID No:8), corresponding to position 349 of SEQ ID No:11 (LRR domain of TM-2-2), by a histidine (H), a lysine (K) or a threonine (T).

The LRR variants according to the invention are thus characterized by the presence of an arginine at the position corresponding to cysteine 848 in the TM-2-2 protein, and/or by a cysteine, a phenylalanine, a methionine, a tyrosine or a tryptophan at the position corresponding to asparagine 822 in the TM-2-2 protein and/or by a histidine, a lysine or a threonine at the position corresponding to serine 825 of the TM-2-2 protein. An LRR variant of the invention may comprise one of the described substitutions, at only one position from 822, 825 and 848, or at two or at all of them. Preferably, an LRR variant of the invention does not comprise simultaneously a C848R and a N822Y substitution.

According to a preferred embodiment, only one of the 822, 825 and 848 positions is substituted. The inventors have moreover shown that a mutation or substitution at position 655 in the LRR variants of the invention may improve the recognition of ToBRFV MP, preferably the substitution of phenylalanine (F) by Leucine (L). Without being bound by theory, it is expected that a mutation at position 655 improves the presentation of the domain of the LRR variants interacting with the ToBRFV MP. Preferred LRR variants of the invention thus also comprise a mutation at position 655, more preferably a F655L mutation. Position 655, with respect to the whole TM2-2 protein, corresponds to position 179 of SEQ ID No:11 (LRR domain of TM-2-2).

The variants of the invention are also characterized by the presence of a tyrosine (Y), a phenylalanine (F) or a tryptophan (W) at the position corresponding to tyrosine 767 in the TM-2-2 protein, i.e. presence of Y, F or W at the position in the LRR variant corresponding to position 291 of the TM-2-2 LRR (SEQ ID No:11). Indeed, as demonstrated by the inventors in the experimental section, said tyrosine, naturally occurring in the TM-2-2 LRR/protein, can be substituted by F or W without loss of function. This position has moreover been demonstrated in Kobayashi et al, as providing the durable resistance to ToMV.

In the following, the numbering of the amino acids is with respect to the position of said amino acids in the full-length TM-2-2 protein (SEQ ID No:8).

The ability of a given LRR domain to confer to NBS-LRR proteins; and especially to a TM-2-2 protein, the capacity to recognize and/or bind at least the Movement Protein of the Tomato Brown Rugose Fruit Virus (ToBRFV), can be tested easily with the transient expression assay disclosed in the experimental section, namely by transient expression in N. benthamiana of a NBS-LRR protein comprising the LRR domain to be tested, in presence of the ToBRFV MP, and by screening for hypersensitive response. Preferably, the NBS-LRR protein is the TM-2-2 protein, in which the naturally occurring LRR domain is substituted by the LRR variant to be tested.

Preferably, a LRR domain mutant of the invention is such that substitution of the LRR domain of TM-2-2, i.e. SEQ ID No:11, by the LRR variant of the invention, retains the capacity to recognize the MP of ToMV, TMV and/or ToMMV, and preferably at least TMV and ToMV.

A variant LRR domain of the invention exhibits at least 90% sequence identity, at the amino acid level, with SEQ ID No: 11, which corresponds to the sequence of the LRR domain of TM-2-2. Sequence identity between two amino acid sequences is as defined usually in the domain of the invention. A suitable program for defining sequence identity is for example Clustal Omega.

According to preferred embodiments of the invention, the LRR variant has preferably at least 95% sequence identity with SEQ ID No:11, preferably at least 96%, at least 97% or at least 98%. According to still further embodiments, the LRR variant has 99% sequence identity with SEQ ID No:11 or more.

Irrespective of the percentage of sequence identity with the wild-type LRR domain of TM-2-2, a variant according to the invention comprises Y, F or W at the position corresponding to position 767 of the TM-2-2 protein and one or more of:
R at the position corresponding to cysteine 848 of the TM-2-2 protein,
C, F, M, Y or W at the position corresponding to asparagine 822 of the TM-2-2 protein, and
H, K or T at the position corresponding to serine 825 of the TM-2-2 protein.

A variant preferably also comprises leucine (L) at the position corresponding to position 655 of the TM-2-2 protein, in addition to the above-mentioned variations.

Variations to the LRR domain of TM-2-2 in the vicinity of amino acids 822, 825 and 848 in the tridimensional structure of the LRR domain are preferably to be limited; amino acids close to positions 822, 825 and 848 are positions 823, 826, 827, 830, 847, 849, 850, 851, 857 and 858. As demonstrated in the examples, some variations at these positions are however acceptable, such as the K857Q substitution.

Variations or mutations between a LRR variant of the invention and SEQ ID No.11 concern thus preferably amino acids which are not those in the vicinity of amino acids 346, 349 and 372 (corresponding to amino acid 822, 825 and 848 in the TM-2-2 protein).

Variations or mutations are preferably conservative amino-acids substitutions, preferably preserving the 3D structure of the LRR domain.

Namely a basic amino acid like lysine or arginine is preferably substituted by another basic amino acid; an acidic amino acid like aspartic acid or glutamic acid is preferably substituted by another acidic amino acid. A small apolar amino acid like glycine, alanine, proline, cysteine or valine is preferably substituted by another small apolar amino acid. A large apolar amino acid like leucine, isoleucine, phenylalanine, methionine or tryptophan is preferably substituted by another large apolar amino acid. A small polar amino acid like serine or threonine is preferably substituted by another small polar amino acid. A large polar amino acid like asparagine or glutamine is preferably substituted by another large polar amino acid. An aromatic amino acid like tyrosine, phenylalanine or tryptophan is preferably substituted aby another aromatic amino acid.

Potential variations or mutations according to the invention may concern amino acids corresponding to position 655 of TM-2-2, or position 857 and/or 769, as demonstrated in the experimental section. According to a preferred embodiment, a LRR domain variant according to the invention exhibits 20 mutations or less with respect to the LRR domain of TM-2-2, preferably 15 or less, and more preferably 10 mutations or less. Said mutations are preferably conservative mutations; they are preferably to be found in portions of the LRR domain which are not in the vicinity of cysteine 848, asparagine 822 and serine 825 substitutions.

Potential LRR variants according to the invention comprise: leucine (L) at position 655, arginine (R) at position 848 and tyrosine (Y) at position 767, or leucine (L) at position 655, arginine (R) at position 848 and phenylalanine (F) at position 767, or leucine (L) at position 655, arginine (R) at position 848 and tryptophan (WW) at position 767, or arginine (R) at position 848 and tyrosine (Y) at position 767, as well as all the LRR variants illustrated in the examples.

The present invention is also directed to a nucleotide sequence encoding a LRR domain mutant as defined above. In view of the degeneracy of the genetic code, very different nucleotide sequences can be envisaged, coding for a LRR domain variant of the invention, namely coding for a variant of SEQ ID No: 11, characterized essentially by an arginine at the position corresponding to cysteine 848 of the TM-2-2 protein, and/or by a cysteine, a phenylalanine, a methionine, a tyrosine or a tryptophan at the position corresponding to asparagine 822 and/or by a histidine, a lysine or a threonine at the position corresponding to serine 825, wherein said variant of SEQ ID No:11 has a tyrosine, a phenylalanine or a tryptophan at position 767, and potentially a leucine at position 655. The sequence can be isolated or not.

A suitable nucleotide sequence is for example a sequence corresponding to SEQ ID No:12, namely the wild type sequence encoding Tm-2-2 LRR, in which at least the codon corresponding to cysteine at position 848 of TM-2-2 has been substituted by a codon corresponding to arginine, or at least the codon corresponding to asparagine at position 822 of TM-2-2 has been substituted by a codon corresponding to cysteine, phenylalanine, methionine, tyrosine or tryptophan, or at least the codon corresponding to serine at position 825 of TM-2-2 has been substituted by a codon corresponding to histidine, lysine or threonine. Suitable nucleotide sequences are for example nucleotides 1429 to 2586 (or 2583 in excluding the stop codon) of SEQ ID No: 4, or 5. Other suitable nucleotide sequences deriving from SEQ ID No:12, in which at least one of the following codons has been substituted:
the codon TGC encoding the cysteine at position 848 of TM-2-2, i.e. the nucleotides at positions 1114-1116 of SEQ ID No:12, has been substituted by a codon encoding an arginine, i.e. by AGA, AGG, CGG, CGA, CGC or CTG,
the codon AAT encoding the asparagine at position 822 of TM-2-2, i.e. the nucleotides at positions 1036-1038 of SEQ ID No:12, has been substituted by a codon encoding a cysteine, i.e. by TGT or TGC, by a codon encoding a phenylalanine, i.e. by TTT or TTC, by a codon encoding a methionine, i.e by ATG, by a codon encoding a tyrosine, i.e. by TAT or TAC, or by a codon encoding a tryptophan, i.e. by TGG, and
the codon TCT encoding the serine at position 825 of TM-2-2, i.e. the nucleotides at positions 1045-1047 of SEQ ID No:12, has been substituted by a codon encoding a histidine, i.e. by CAT or CAC, by a codon encoding a lysine, i.e. by AAA or AAG, or by a codon encoding a threonine, i.e. by ACC, ACT, ACG or ACA.

In addition, the codon TAC encoding the tyrosine at position 767 of TM-2-2, i.e. the nucleotides at positions 871-873 of SEQ ID No.:12, may be substituted by a codon encoding a phenylalanine, i.e. by TTT or TTC or by a codon encoding a tryptophan, i.e. by TGG.

The codon encoding the phenylalanine at position 655 of TM-2-2, i.e. the nucleotides at positions 535-537 of SEQ ID No.:12, may be substituted by a codon encoding a leucine, i.e. by CTT, CTC, CTA, CTG, TTA or TTG.

The present invention also concerns sequences deriving from SEQ ID No:12 having at least 70% sequence identity with said sequence, preferably at least 75%, at least 80%, at least 85%, at least 90% or at least 95% sequence identity. Irrespective of the percentage of sequence identity with SEQ ID No:12, a sequence according to the invention encodes a LRR domain variant of the invention as defined above. Given the degeneracy of the genetic code, a sequence encoding a LRR domain variant of the invention may nevertheless have less than 70% sequence identity with SEQ ID No:12, and is also in the scope of the present invention.

According to a further aspect, the invention is also directed to polypeptides and proteins comprising a mutant LRR domain of the invention, especially polypeptides and proteins, recognizing or binding the Movement Protein (MP) of at least ToBRFV, and preferably also of other tobamoviruses including at least ToMV or TMV, and preferably also ToMMV.

According to a preferred embodiment of the present invention, a protein comprising a mutant or variant LRR domain of interest is a member of the NLR class of protein (NBS-LRR), and more preferably is a coiled-coil NLR (CC-NBS-LRR). As demonstrated in Slootweg et al, LRR exchange between NBS-LRR proteins may indeed modify the target of the NBS-LRR protein. The LRR domain mutant of the invention can thus be substituted to the wild-type LRR of any NBS-LRR thus creating a chimeric NBS-LRR specifically targeting the MP of ToBRFV, and preferably also the MP of ToMV, TMV and/or ToMMV.

The present invention thus also encompasses such chimeric NBS-LRR proteins, comprising the NBS portion of a NBS-LRR protein, and the LRR domain mutant according to the invention. As indicated above, such a NBS-LRR protein is preferably a CC-NBS-LRR.

Such a NBS-LRR protein of the invention preferably comprises the NBS portion of the TM-2-2 protein, corresponding to amino acids 1 to 476 of SEQ ID No:8, fused to a LRR domain mutant of the invention. Such a protein may advantageously have the sequence SEQ ID No:9 (TM2-14-25), SEQ ID No:10 (TM2-467), SEQ ID No:17 (TM2-4), SEQ ID No:18 (TM2-5), SEQ ID No:19 (TM2-825H), SEQ ID No:20 TM2-825K), SEQ ID No:21 (TM2-825T), SEQ ID No:22 (TM1-822C), SEQ ID No:23 (TM2-822F), SEQ ID No:24 (TM2-822M), SEQ ID No:25 (TM2-822Y) or SEQ ID No:26 (TM2-822-W). These proteins of the invention are thus TM-2-2 protein variants, differing from the TM-2-2 protein only in the LRR domain.

Alternative NBS-LRR proteins of the invention comprise the NBS portion of SEQ ID No:6 and 7 (corresponding to the proteins encoded by tm2 and Tm2 genes) fused to a LRR domain mutant of the invention.

The invention is also directed to TM-2-2 protein variants, having at least 90% amino acid identity with the sequence of the TM-2-2 protein (SEQ ID No:8), interacting with and/or recognizing or targeting the MP of ToBRFV and comprising a tyrosine, a phenylalanine or a tryptophan at the position corresponding to Y767 of SEQ ID No:8, and comprising at least one of an arginine at position corresponding to C848 of SEQ ID No:8, a histidine, a lysine or a threonine at the position corresponding to S825 and a cysteine, a phenylalanine, a methionine, a tyrosine or a tryptophan at the position corresponding to N822, i.e. comprising at least one of the following variations: N822C, N822F, N822M, N822Y, N822W, S825H, S825K, S825T and C848R. Such variants may also comprise a leucine at the position corresponding to F655 of SEQ ID No:8. The variations within the TM-2-2 protein may be present in the NBS domain or in the LRR domain of the protein. They are preferably to be found mainly in the NBS domain. For variations in the LRR domain, the requirements for the variations or mutations are as described previously in connection with the LRR mutants of the invention. The variations or mutations are preferably conservative mutations. The variants are isolated or not.

According to a preferred embodiment, only one of the 822, 825 and 848 positions is substituted. According to alternative embodiments, two of said positions, or all these positions are substituted. Preferably, the variant does not comprise simultaneously C848R and N822Y substitutions.

The TM-2-2 protein variants according to the invention have preferably at least 90% amino acid identity globally with the sequence of the TM-2-2 protein, with at least 95% sequence identity at the LRR domain level. According to additional embodiments, the global percentage of sequence identity of the TM-2-2 protein variants of the invention is at least 95% with SEQ ID No:8, with preferably a greater sequence identity at the LRR domain level. Preferably, the sequence identity between a TM-2-2 protein and SEQ ID No:8 is at least 96%, at least 97%, at least 98% or even at least 99%.

According to a preferred embodiment, a TM-2-2 protein variant according to the invention exhibits 60 mutations or less with respect to the TM-2-2 protein, preferably 50 or less, and more preferably 20 mutations or less. Said mutations are preferably conservative mutations; they are preferably to be found in majority in the domains of the protein which are not the LRR domain.

A TM-2-2 protein variant according to the invention triggers a hypersensitive response (HR) in presence of the MP of ToBRFV, and preferably also in presence of MP of other tobamoviruses, including at least one of TMV, ToMV and ToMMV. A TM-2-2 protein variant according to the invention thus confers resistance against ToBRFV infection in tomatoes, and preferably also resistance against additional tobamoviruses, including TMV, ToMV and/ToMMV.

It is noted in this regard that detection of a hypersensitive response in a transient expression assay in a surrogate plant like N. benthamiana equates detection of resistance in tomato. There is indeed a perfect correlation between the ability of a NBS-LRR protein to trigger a robust HR response in N. benthamiana leaves (in the presence of a tobamovirus MP) and virus resistance in tomato. This point has been confirmed by the inventors in the experimental section.

According to still another embodiment, the invention is also directed to a nucleotide sequence encoding a polypeptide or protein as disclosed above, especially encoding a protein comprising a LRR domain mutant of the invention, a chimeric NBS-LRR as described or a TM-2-2 protein variant of the invention. A nucleotide sequence according to the invention encompasses at least DNA, single-stranded DNA, RNA, double stranded RNA, and mixture of DNA and RNA. The sequence can be isolated or not.

A suitable nucleotide sequence is for example SEQ ID No:4 or 5; these sequences are derived from the wild type sequence Tm-2-2 encoding TM-2-2, in which at least the codon corresponding to cysteine at position 848 of TM-2-2 has been substituted by a codon corresponding to arginine. The present invention is thus also directed to mutated Tm-2-2 genes, as for example SEQ ID No:4 (Tm2-14-25) or SEQ ID No:5 (Tm2-467). The invention is also directed to a sequence deriving from SEQ ID No:3, corresponding to the wild type sequence Tm-2-2 encoding TM-2-2, in which at least one of the following codons has been substituted:
the codon TGC encoding the cysteine at position 848 of TM-2-2, i.e. the nucleotides at positions 2542-2544 of SEQ ID No:3, has been substituted by a codon encoding an arginine, i.e. by AGA, AGG, CGG, CGA, CGC or CTG,
the codon AAT encoding the asparagine at position 822 of TM-2-2, i.e. the nucleotides at positions 2464-2466 of SEQ ID No:3, has been substituted by a codon encoding a cysteine, i.e. by TGT or TGC, by a codon encoding a phenylalanine, i.e. by TTT or TTC, by a codon encoding a methionine, i.e by ATG, by a codon encoding a tyrosine, i.e. by TAT or TAC, or by a codon encoding a tryptophan, i.e. by TGG, and/or
the codon TCT encoding the serine at position 825 of TM-2-2, i.e. the nucleotides at positions 2473-2475 of SEQ ID No:3, has been substituted by a codon encoding a histidine, i.e. by CAT or CAC, by a codon encoding a lysine, i.e. by AAA or AAG, or by a codon encoding a threonine, i.e. by ACC, ACT, ACG or ACA.

The codon TAC encoding the tyrosine at position 767 of TM-2-2, i.e. the nucleotides at positions 2299-2301 of SEQ ID No:3 may also be substituted by a codon encoding a phenylalanine, i.e. by TTT or TTC, or by a codon encoding a tryptophan, i.e. by TGG.

The codon TTT encoding the phenylalanine at position 655 of TM-2-2, i.e. the nucleotides at positions 1963-1965 of SEQ ID No:3 may also be substituted by a codon encoding a leucine, i.e. by CTT, CTC, CTA, CTG, TTA or TTG.

According to specific embodiments, a substituted sequence deriving from SEQ ID No:3 as described above is chosen from SEQ ID No:27 (encoding TM2-4), SEQ ID No:28 (encoding TM2-5), SEQ ID No:29 (encoding TM2-825H), SEQ ID No:30 (encoding TM2-825K), SEQ ID No:31 (encoding TM2-825T), SEQ ID No:32 (encoding TM1-822C), SEQ ID No:33 (encoding TM2-822F), SEQ ID No:34 (encoding TM2-822M), SEQ ID No:35 (encoding TM2-822Y) or SEQ ID No:36 (encoding TM2-822-W).

The invention also concerns sequences deriving from SEQ ID No:3, 4, 5, 27, 28, 29, 30, 31, 32, 33, 34, 35 or 36, or from the substituted sequences described above, having at least 70% sequence identity with said sequences, preferably at least 75%, at least 80%, at least 85%, at least 90% or at least 95% sequence identity. Irrespective of the percentage of sequence identity with SEQ ID No:3, 4, 5, 27, 28, 29, 30, 31, 32, 33, 34, 35 or 36, or with the substituted sequences described above, such a sequence encodes a protein, polypeptide or TM-2-2 variant according to the invention. Given the degeneracy of the genetic code, a sequence encoding such a polypeptide, protein or variant of the invention may nevertheless have less than 70% sequence identity with SEQ ID No:3, 4, 5, 27, 28, 29, 30, 31, 32, 33, 34, 35 or 36, or with the substituted sequences described above, but is also in the scope of the present invention.

Potential additional variations are especially substitution of Y767 by F or W. Additional mutations or variations have already been explicated in the preceding aspects of the invention are applied mutatis mutandis to this aspect.

The invention according to another aspect also relates to a Resistance gene, encoding a NBS-LRR protein which is a mutant or allele of the TM-2-2 protein, conferring to plants, especially tomatoes, resistance against at least ToBRFV, and preferably against at least one or more of TMV, ToMV and ToMMV. Preferably, such a resistance gene confers resistance against at least ToBRFV, TMV and ToMV, and even preferably against ToBRFV, TMV, ToMV and ToMMV. The mutant or allele of the TM-2-2 protein is a variant of the TM-2-2 protein as disclosed above, comprising at least one of the substitutions C848R at position 848 of the TM-2-2 protein, N822C, N822F, N822M, N822Y and N822W at position 822 of the TM-2-2 protein and S825H, S825K and S825T at position 825 of the TM-2-2 protein.

The mutant or allele of the TM-2-2 protein may comprise one of the described substitutions, at only one position from 822, 825 and 848, or at two or at all of them. Preferably, a mutant or allele of the TM-2-2 protein of the invention does not comprise simultaneously a C848R and a N822Y substitution.

The mutant or allele of the TM-2-2 protein may further comprise a F655L substitution.

The newly discovered resistance protein and the resistance gene encoding said protein, confer resistance against ToBRFV, preferably in addition to resistance against ToMV and TMV, thanks to the substitution of the cysteine 848, the asparagine 822 and/or the serine 825 in the LRR domain of the TM-2-2 protein, by an arginine for C848, by a cysteine, a phenylalanine, a methionine, a tyrosine or a tryptophan for N822 and a histidine, a lysine or a threonine for S825.

According to still another aspect, the invention is also directed to a nucleic acid construct comprising a sequence encoding a polypeptide or protein according to the invention or comprising a nucleotide sequence encoding a TM-2-2 variant or comprising a resistance gene according to the invention. Such a sequence, encoding a polypeptide or protein according to the invention, or comprising a nucleotide sequence encoding a TM-2-2 variant or comprising a resistance gene is interchangeably referred to as a nucleotide sequence or resistance gene, or variant Tm-2-2 gene or mutated Tm-2-2 gene of the invention, in the following.

Such a sequence encoding the protein, polypeptide or variant of interest is preferably under the control of a promoter, which is a constitutive or inducible promoter. Preferably, the promoter is a promoter which is active in plant cells. According to an embodiment, the promoter is not the wild-type promoter of Tm-2, Tm-2-2 or tm-2 gene. Preferably the promoter for the Tm-2-2 variant gene is the native promoter of Tm-2, Tm-2-2 or tm-2.

A nucleic acid construct according to the invention can thus be a vector, a plasmid or a T-DNA plasmid. Presence of a construct of the invention in a cell thus may give rise to the expression of a protein, polypeptide TM-2-2 variant or Resistance protein according to the invention and as defined above.

The invention also encompasses expression vector or construct suitable for expression of a polypeptide, protein, TM-2-2 variant or resistance protein according to the invention, preferably expression in a plant cell.

According to a further embodiment, the invention is also directed to the use of a sequence as defined, encoding a TM-2-2 variant of the invention, or a construct comprising such a sequence for conferring resistance against at least ToBRFV to a S. lycopersicum plant or for obtaining transgenic S. lycopersicum plants resistant against ToBRFV. Indeed, as demonstrated in the example, the TM-2-2 variant recognizes the ToBRFV MP thus triggering a HR response associated with ToBRFV resistance. A TM-2-2 variant of the invention may also recognize at least the movement proteins of TMV, ToMV and/or ToMMV, thus conferring resistance against these tobamoviruses. The invention thus also encompasses the use of said sequence for conferring resistance against ToBRFV and against at least one of TMV, ToMV and ToMMV, to a S. lycopersicum plant and also uses for obtaining transgenic S. lycopersicum plants exhibiting these resistances.

According to a further aspect, the invention also concerns a cell comprising a nucleotide sequence or resistance gene according to the invention or a DNA construct as disclosed above.

The cell is preferably a plant cell, preferably from the Solanaceae family, for example from the Solanum genus, and even more preferably a cell of S. lycopersicum plant, or a cell of the Capsicum or Nicotiana genus. The cell comprises, in its genome and preferably in its nuclear genome, a nucleotide sequence or resistance gene or DNA construct according to the present invention. The presence of these sequences confers the phenotype of interest, namely expression of a protein interacting with at least the ToBRFV MP triggering HR response in suitable conditions. The presence of these sequences can be revealed by any techniques well known to the skilled reader, based on the sequence.

Particularly preferred types of cells are cells of the Solanum, Nicotiana or Capsicum genus, and more preferably S. lycopersicum cells.

Cells according to the invention can be any type of cell, especially of S. lycopersicum cell, inter alia an isolated cell and/or a cell capable of regenerating a whole plant, especially a S. lycopersicum plant bearing the nucleotide sequence or resistance gene of the invention. A cell can thus be a regenerable cell or a non regenerable cell.

The nucleotide sequence or resistance gene of interest can be present homozygously or heterozygously in a cell of the invention. Preferably, a cell according to the invention comprises the resistance gene, or the nucleotide sequence as defined above, at the heterozygous state.

The present invention is also directed to a tissue culture of non-regenerable or regenerable cells as defined above according to the present invention; preferably, the regenerable cells are derived from embryos, protoplasts, meristematic cells, callus, pollen, leaves, anthers, stems, petioles, roots, root tips, fruits, seeds, flowers, cotyledons, and/or hypocotyls of the invention, and the cells contain the nucleotide sequences or resistance gene in their genome conferring the resistance against at least ToBRFV. Preferably, such sequences or resistance gene also provide resistance against TMV and/or ToMV, and preferably also against ToMMV.

The invention is also directed to any plant part, especially a S. lycopersicum plant part, particularly seeds, explants, reproductive material, scion, cutting, seed, fruit, root, rootstock, pollen, ovule, embryo, protoplast, leaf, anther, stem, petiole or flowers, wherein said plant part comprises at least one cell as described above.

The invention also provides a protoplast comprising the nucleotide sequences or resistance gene of the invention.

According to another aspect, the invention is directed to a plant, and more preferably to a S. lycopersicum plant comprising in its genome a nucleotide sequence or a resistance gene as defined above, encoding a protein or peptide of the invention. Such a nucleotide sequence or resistance gene thus encodes for a variant of the TM-2-2 protein, comprising (1) at least one of a C848R mutation at position 848, a N822C, N822F, N822M, N822Y or N822W mutation at position 822, and a S825H, S825K or S825T mutation at position 825, and (2) Y, F or W at position 767, and conferring resistance against at least ToBRFV, and preferably also against one or more of TMV, ToMV and ToMMV, and more preferably against TMV, ToMV and ToMMV, in addition to ToBRFV. Preferably the nucleotide sequence or resistance gene encodes a variant of the TM-2-2 protein further comprising leucine at position 655.

The invention thus also encompasses a S. lycopersicum plant resistant against at least ToBRFV, comprising a mutated Tm-2-2 gene encoding a variant of the TM-2-2 protein (SEQ ID No:8) according to the invention namely having at least 90% sequence identity with SEQ ID No:8, comprising a tyrosine, phenylalanine or tryptophan at the position corresponding to position 767 of SEQ ID No:8, and comprising at least one of the mutations/variations:
an arginine at the position corresponding to position 848 of SEQ ID No:8, instead of the cysteine present in the wild type TM-2-2 sequence;
a cysteine, a phenylalanine, a methionine, a tyrosine or a tryptophan at the position corresponding to position 822 of SEQ ID No:8, instead of the asparagine present in the wild type TM-2-2 sequence, and
a histidine, a lysine or a threonine at the position corresponding to position 825 of SEQ ID No:8, instead of the serine present in the wild type TM-2-2 sequence.

The mutated Tm-2-2 gene is a resistance gene according to the invention.

It may advantageously also comprise a leucine at the position corresponding to position 655 of SEQ ID No:8, instead of the phenylalanine present in the wild-type TM-2-2 sequence.

The resistance gene or nucleotide sequence of the invention is preferably stably present in the nuclear genome of the cells of the plant. It can be either stably integrated into the nuclear genome, for example after transformation, or it can result from a mutagenesis process such as Tilling as detailed in the experimental section of the application. The presence of these sequences conferring at least resistance against ToBRFV may also result from introgression from a resistant parent. These sequences conferring resistance against tobamoviruses, including at least ToBRFV, are preferably but not necessarily, to be found on chromosome 9, at the locus of the tm-2 or the Tm-2 gene. Other positions in the genome, for example resulting from random integration, are also suitable and encompassed by the present invention.

The resistance phenotype can be tested and scored as described in the experimental section, especially in example 1.4, by natural infection or by artificial inoculation, at the first leaves level, or at the fruit level.

The sequences or resistance gene conferring resistance against at least ToBRFV can be present homozygously or heterozygously in the genome of a plant according to the invention, especially a S. lycorpersicum plant. These sequences can also be present as multiple copies.

The resistance gene or mutated Tm-2-2 gene according to this aspect of the invention is as defined in connection with the other aspects of the invention and thus encodes a variant of TM-2-2 protein, which may be SEQ ID No:9, SEQ ID No:10, SEQ ID No:17, SEQ ID No:18, SEQ ID No:19, SEQ ID No:20, SEQ ID No:21, SEQ ID No:22, SEQ ID No:23, SEQ ID No:24, SEQ ID No:25 or SEQ ID No:26. Suitable resistance genes or mutated Tm-2-2 genes are for example those having SEQ ID No:4 or SEQ ID No:5, SEQ ID No:27, SEQ ID No:28, SEQ ID No:29, SEQ ID No:30, SEQ ID No:31, SEQ ID No:32, SEQ ID No:33, SEQ ID No:34, SEQ ID No:35, SEQ ID No:36, or the substituted sequences described above, or sequences derived from SEQ ID No:4 or SEQ ID No:5 or from the substituted sequences described above further to the degeneracy of the genetic code.

The invention is also directed to tissue of a plant of the invention; the tissue can be an undifferentiated tissue, or a differentiated tissue. Such a tissue comprises one or more cells comprising the resistance gene or mutated Tm-2-2 gene of the invention.

The invention is also directed to propagation material, capable of producing a resistant plant according to the invention, especially a resistant tomato plant, comprising the resistance gene or mutated Tm-2-2 gene as defined above. The invention is particularly directed to seeds of such a resistant plant, comprising the resistance gene or mutated Tm-2-2 gene, especially S. lycopersicum seeds and seed which can be grown into a S. lycopersicum plant according to the invention.

The seed of such S. lycopersicum are preferably coated or pelleted with individual or combined active species such as plant nutrients, enhancing microorganisms, or products for disinfecting the environment of the seeds and plants. Such species and chemicals may be a product that promotes the growth of plants, for example hormones, or that increases their resistance to environmental stresses, for example defense stimulators, or that stabilizes the pH of the substrate and its immediate surroundings, or alternatively a nutrient.

They may also be a product for protecting against agents that are unfavorable toward the growth of young plants, including herein viruses and pathogenic microorganisms, for example a fungicidal, bactericidal, hematicidal, insecticidal or herbicidal product, which acts by contact, ingestion or gaseous diffusion; it is, for example, any suitable essential oil, for example extract of thyme. All these products reinforce the resistance reactions of the plant, and/or disinfect or regulate the environment of said plant. They may also be a live biological material, for example a nonpathogenic microorganism, for example at least one fungus, or a bacterium, or a virus, if necessary with a medium ensuring its viability; and this microorganism, for example of the pseudomonas, bacillus, trichoderma, clonostachys, fusarium, rhizoctonia, etc. type stimulates the growth of the plant, or protects it against pathogens.

A plant, cell or seed of the invention may be heterozygous or homozygous for the resistance gene or mutated Tm-2-2 gene of the invention conferring ToBRFV resistance. The resistance imparted by this gene is expected to be dominant, such that plants having heterozygously the resistance gene or mutated Tm-2-2 gene of the invention are also resistant to ToBRFV. The present invention thus also encompasses plant, cell or seed having heterozygously in their genome the resistance gene or mutated Tm-2-2 gene of the invention as defined above.

Preferably, a S. lycopersicum plant according to the invention is a commercial plant or line. Such a commercial plant or line preferably also exhibits one or more of the following additional features: nematode resistance trait (Mi-1 or Mi-j), Fusarium resistance, Verticillium resistance, and/or TYLCV resistance.

Other resistances or tolerances are also envisaged according to the invention.

Moreover, the commercial plant of the invention gives rise to fruits in suitable conditions, which are at least 10 grams, preferably 25 grams at full maturity, preferably at least 100 g at full maturity and or even more preferably at least 150 g or at least 200 g at full maturity. The number of fruits per plant is moreover essentially unaffected by the presence of the resistance gene or mutated Tm-2-2 gene of the invention, i.e. the productivity of a plant according to the invention is not inferior by more than 20% to a plant having the same genotype but devoid of said resistance gene or mutated Tm-2-2 gene.

According to still another embodiment, a plant of the invention is a determinate, indeterminate or semi-indeterminate plant, or seed or cell thereof, i.e. corresponding to determinate, indeterminate or semi-indeterminate growth habit.

By determinate, it is meant tomato plants which tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties.

A plant, cell or seed according to the present invention may advantageously also comprise the Tm-1 gene. The Tm-1 gene is as defined inter alia in the publication Ishibashi et al, 2007; preferably ‘Tm-1 gene’ refers to a genetic sequence encoding a protein having the Tm-1 activity reported in the article, namely the ability to inhibit the viral replication of a wild-type ToMV strain Tm-1 sensitive, for example the strain ToMV-L disclosed in this article.

The invention thus also encompasses tomato plant, cell or seed comprising the Tm-1 gene, either homozygously or heterozygously, in addition to the resistance gene or mutated Tm-2-2 gene of the invention.

According to still another embodiment, a plant of the invention is used as a scion or as a rootstock in a grafting process. Grafting is a process that has been used for many years in crops such as cucurbitacea, but only more recently for tomato. Grafting may be used to provide a certain level of resistance to telluric pathogens such as Phytophthora or to certain nematodes. Grating is therefore intended to prevent contact between the plant or variety to be cultivated and the infested soil. The variety of interest used as the graft or scion, optionally an F1 hybrid, is grafted onto the resistant plant used as the rootstock. The resistant rootstock remains healthy and provides, from the soils, the normal supply for the graft that it isolates from the diseases.

As detailed above, the invention is directed to S. lycopersicum plants, exhibiting the ToBRFV resistance, preferably also TMV, ToMV and/or ToMMV resistance, as well as to seeds giving rise to those plants, and cells of these plants or seeds, or other plant parts, comprising the resistance gene or mutated Tm-2-2 gene in their genome, and to progeny of such a plant of the invention comprising said resistance gene or mutated Tm-2-2 gene.

Progeny encompasses the first, the second, and all further descendants from a cross with a plant according to the invention, wherein a cross comprises a cross with itself or a cross with another plant.

It is noted that the seeds or plants of the invention may be obtained by different processes, and are not exclusively obtained by means of an essentially biological process. The resistance gene or the mutated Tm-2-2 gene can indeed be introduced, incorporated or obtained “in cellulo” by different techniques. Plants, cells or seeds according to the invention may thus be transgenic, or non-transgenic, they are preferably obtained by technical processes which are not essentially biological processes, as detailed in other sections of this description and in the examples. According to a preferred embodiment, the plants, cells or seeds according to the invention are not exclusively obtained by means of an essentially biological process.

A mutated Tm-2-2 gene, encoding a TM-2-2 variant as disclosed, may advantageously be obtained by gene editing techniques, base-editing or prime editing techniques, such a mutagenesis, especially targeted mutagenesis such a TILLING, or by other gene editing techniques such as CRISPR/Cas system, or by custom-made endonucleases, or by base-editing or prime editing with Cas9, Cas12a or other Cas proteins.

These techniques are well known to the skilled reader. Examples of these techniques are illustrated in the experimental section of the application.

In specific embodiments of the invention, the mutations in the Tm-2-2 gene are induced by means of genetic engineering. The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated through the use of Zinc finger nuclease (ZFN) technology (ZFN-1, ZFN-2 and ZFN-3, see U.S. Pat. No. 9,145,565), Oligonucleotide directed mutagenesis (ODM), Cisgenesis and intragenesis, Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease re-engineered homing endonucleases, DNA guided genome editing (Gao et al., Nature Biotechnology (2016)), and Synthetic genomics. A major part of targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”.

A resistance gene as defined may also be introduced into a plant, cell or seed of the invention by transformation, especially Agrobacterium transformation, thus producing transgenic plants, cells or seeds. This technique is also illustrated in the example section of the application.

The application is directed to plants, seed or cells comprising the resistance gene or the mutated Tm-2-2 gene as already defined irrespective of the mode of provision of these sequences; and thus is directed to transgenic and non-transgenic plants.

In a further aspect, the invention is also directed to different methods for obtaining, breeding or producing plants, especially S. lycopersicum plant resistant against ToBRFV, and preferably also resistant against ToMV, TMV and/or ToMMV, more preferably resistant against ToMV, TMV and ToMMV.

The invention thus encompasses methods of producing a plant, especially S. lycopersicum plant resistant to ToBRFV, and preferably also resistant to ToMV, TMV and/or ToMMV comprising the following steps:
a) treating M0 seeds of a plant, preferably a tomato plant to be modified with a mutagenic agent to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M1+n seeds.

The M1 or M2 seeds are grown into plants and submitted to ToBRFV infection or to screening to identify variations in the Tm-2-2 gene.

In this method, the M1 seeds of step a) can be obtained via chemical mutagenesis such as EMS mutagenesis or by other chemical mutagenic agents or physical means, such as irradiation, which is for example selected ionizing radiations (X-Ray, gamma rays, alpha particles . . . ), heavy-ion beam irradiation, ultraviolet radiations, radioactive decay or fast neutrons irradiation.

Another method for producing a S. lycopersicum plant resistant against at least ToBRFV, and preferably also against one or more of TMV, ToMV and ToMMV, comprises the introduction into a plant already comprising a tm2, Tm-2 or Tm-2-2 gene on chromosome 9, of mutations into said tm2, Tm-2 or Tm-2-2 gene, in order to create a mutated Tm-2-2 gene according to the invention, namely encoding a TM-2-2 variant comprising at least one of the C848R, N822C, N822F, N822M, N822Y, N822W, S825H, S825K and S825T mutations with respect to TM-2-2 and comprising F, Y or W at position 767, and potentially also comprising the F655L mutation.

When the starting material is a S. lycopersicum plant comprising a Tm-2-2 gene, the method advantageously comprises the introduction of at least one mutation in said Tm-2-2 gene, preferably by mutagenesis, by TILLING or by genome editing, base-editing or prime editing, in particular by mutagenesis induced by a physical agent or a chemical agent, inter alia by a technique selected from ethyl methanesulfonate (EMS) mutagenesis, N-methyl-N-nitrosourea (MNU) mutagenesis, or Sodium Azide (NaN3, SA) mutagenesis, oligonucleotide directed mutagenesis (ODM), Zinc finger nuclease (ZFN) technology, Transcription Activator-Like Effector Nucleases (TALENs), the CRISPR/Cas system, Cas9, Cas12a, or other Cas proteins, engineered meganuclease, re-engineered homing endonucleases and DNA guided genome editing, wherein said at least one mutation gives rise to the C848R, N822C, N822F, N822M, N822Y, N822W, S825H, S825K or S825T substitution in the protein encoded by the Tm-2-2 gene. The method may comprise the introduction of further mutations in the Tm-2-2 gene, for example for giving rise to the Y767F or Y767W substitution in the protein encoded by the Tm-2-2 gene.

Additional mutations may also be introduced, provided the recognition of the ToBRFV MP is not lost, and preferably also the recognition of the TMV, ToMV and ToMMV MPs is also not lost.

The invention also encompasses different methods for obtaining transgenic plants according to the invention, especially transgenic S. lycopersicum plants resistant against al least ToBRFV and preferably also resistant against ToMV, TMV and/or ToMMV, by introducing a resistance gene or mutated Tm-2-2 gene according to the invention. These methods may comprise the following steps:
Obtaining a DNA construct as defined in a preceding aspect of the invention, i.e. comprising a resistance gene or mutated Tm-2-2 gene, encoding a TM-2-2 variant according to the invention,
Introducing said construct into a cell, especially into a S. lycopersicum cell,
Regenerating a transgenic plant and
Optionally propagating the obtained plant.

According to another aspect, the present invention is also directed to the use of a tomato seed or plant of the invention, preferably comprising homozygously the resistance gene or mutated Tm-2-2 gene of the invention, as a breeding partner in a breeding program for obtaining S. lycopersicum plants having the ToBRFV resistance phenotype, and preferably having ToBRFV, ToMV, TMV and ToMMV resistances. Indeed, such a breeding partner harbors homozygously in its genome the resistance gene or mutated Tm-2-2 gene conferring the phenotype of interest. By crossing this plant with a tomato plant, especially a line, it is thus possible to transfer the resistance gene or mutated Tm-2-2 gene of the present invention conferring the desired phenotype, to the progeny. A plant according to the invention can thus be used as a breeding partner for introgressing the resistance gene or mutated Tm-2-2 gene into a S. lycopersicum plant or germplasm. Although a plant or seed bearing heterozygously the resistant gene of interest, can also be used as a breeding partner as detailed above, the segregation of the phenotype is likely to render the breeding program more complex.

The invention thus also concerns a method for breeding a S. lycopersicum plant resistant against at least ToBRFV and preferably also to TMV, ToMV and/or ToMMV, most preferably to all of them, comprising:
(a) Crossing a S. lycopersicum plant comprising a resistance gene or mutated Tm-2-2 gene according to the invention with an initial S. lycopersicum plant devoid of resistance gene or mutated gene,
(b) Selecting in the progeny thus obtained, a plant bearing the resistance gene or mutated gene,
(c) Optionally self-pollinating one or several times the plant obtained at step (b) and selecting in the progeny thus obtained a plant bearing the resistance gene or mutated gene.

The selection can be made by any appropriate means well known to the skilled person, inter alia by using markers specific to the resistance gene or mutated gene.

The invention is also directed to a method of producing a S. lycopersicum plant resistant to at least ToBRFV, and preferably also to TMV, ToMV and/or ToMMV, most preferably to all of them, comprising obtaining a part of a plant according to the invention, thus comprising a resistance gene or mutated Tm-2-2 gene as already defined, and vegetatively propagating said plant part to generate a plant from said plant part.

In all the methods and processes according to the invention, the S. lycopersicum plant is determinate, indeterminate or semi-determinate.

As already disclosed, the tomato plants according to the invention are preferably also resistant to nematodes, TYLCV, Fusarium and/or Verticillium.

The present invention is also directed to a S. lycopersicum plant and seed obtained or obtainable by any of the methods and processes disclosed above. Such a plant is indeed a S. lycopersicum plant expressing a TM-2-2 variant of the invention conferring resistance against tobamoviruses, especially against ToBRFV, and preferably also to at least one of TMV, ToMV and ToMMV.

According to still another aspect, the invention is also directed to a method for genotyping a plant, preferably a S. lycopersicum plant or tomato germplasm, for the presence of a resistance gene or mutated Tm-2-2 gene according to the invention associated with resistance against ToBRFV infection, wherein the method comprises the determination or detection in the genome of the tested plant of a nucleic acid comprising or corresponding to at least one of the C848R, N822C, N822F, N822M, N822Y, N822W, S825H, S825K and S825T mutations in the Tm-2-2 gene. Preferably, the method comprises the step of identifying in a sample of the plant to be tested specific sequences associated with one of these mutations conferring resistance to ToBRFV. Similarly, the invention is also directed to a method for identifying, detecting and/or selecting S. lycopersicum plants resistant to ToBRFV in plants resistant to TMV and ToMV, said method comprising the detection of a mutant allele of the Tm-2-2 gene in the genome of said plants, wherein said mutant allele comprises at least one mutation chosen from the mutations giving rise to a C848R amino acid substitution at position 848 of the TM-2-2 protein, the mutations giving rise to a N822C, N822F, N822M, N822Y or N822W amino acid substitution at position 822 of the TM-2-2 protein and the mutations giving rise to a S825H, S825K and S825T amino acid substitution at position 825 of the TM-2-2 protein.

In view of the ability of the resistant plants of the invention to restrict the damages caused by different tobamoviruses infection, including ToBRFV infection, they are advantageously grown in an environment infested or likely to be infested or infected by ToBRFV, and potentially also by TMV, ToMV and/or ToMMV; in these conditions, the resistant plants of the invention produce more marketable tomatoes than susceptible plants. The invention is thus also directed to a method for improving the yield of tomato plants in an environment infested by ToBRFV, and also potentially by TMV, ToMV and/or ToMMV, comprising growing tomato plants comprising in their genome the resistance gene or mutated Tm-2-2 gene as defined according to the previous aspects of the invention, and conferring to said plants resistance to at least ToBRFV.

Preferably, the method comprises a first step of choosing or selecting a tomato plant comprising said resistance gene or mutated Tm-2-2 gene of interest. The method can also be defined as a method of increasing the productivity of a tomato field, tunnel or glasshouse, or as a method of reducing the intensity or number of chemical or fungicide applications in the production of tomatoes.

The invention is also directed to a method for reducing the loss on tomato production in condition of ToBRFV infestation or infection, or more generally in condition of ToMV, TMV and/or ToBRFV infestation, comprising growing a tomato plant as defined above.

These methods are particularly valuable for a population of tomato plants, either in a field, in tunnels or in glasshouses.

Alternatively, said methods for improving the yield or reducing the loss on tomato production may comprise a first step of identifying tomato plants resistant to ToBRFV, preferably also to ToMV, TMV and ToMMV, and comprising in their genome the resistance gene or mutated Tm-2-2 gene of the invention, that confers to said plants at least ToBRFV resistance, and then growing said resistant plants in an environment infested or likely to be infested by the virus.

The resistant plants of the invention are also able to restrict the growth of ToBRFV, thus limiting the infection of further plants and the propagation of the virus. Accordingly, the invention is also directed to a method of protecting a field, tunnel or glasshouse, or any other type of plantation, from ToBRFV infection, or of at least limiting the level of infection by ToBRFV of said field, tunnel or glasshouse or of limiting the spread of ToBRFV in a field, tunnel or glasshouse, especially in a tomato field. Such a method preferably comprises the step of growing a resistant plant of the invention, i.e. a plant comprising in its genome the resistance gene or mutated Tm-2-2 gene, conferring to said plant ToBRFV resistance.

The invention also concerns the use of a plant resistant to ToBRFV for controlling ToBRFV infection or infestation in a field, tunnel or glasshouse, or other plantation; such a plant is a plant of the invention, comprising in its genome the resistance gene or mutated Tm-2-2 gene as defined above. This use or method is also a method for disinfecting a field, tunnel or glasshouse by decreasing its viral population.

In still a further aspect, the invention also relates to a method of producing tomatoes comprising:
a) growing a S. lycopersicum plant of the invention, comprising the resistance gene or mutated Tm-2-2 gene as defined previously;
b) allowing said plant to set fruit; and
c) harvesting fruit of said plant, preferably at maturity and/or before maturity.

All the preferred embodiments regarding the resistance gene or mutated Tm-2-2 gene are already disclosed in the context of the previous aspects of the invention. The method may advantageously comprise a further step of processing said tomatoes into a tomato processed food.

The invention also concerns a process for the production of tomatoes in a transgenic tomato plant, comprising introducing into a S. lycopersicum plant a nucleic acid molecule coding for a TM-2-2 variant according to the invention. The process may further comprise a step of regenerating a transgenic plant, and allowing the plant to set fruit. The process may also comprise a step of harvesting fruit(s) of said transgenic plant.

According to still another embodiment, the invention is also directed to a method for identifying, detecting and/or selecting mutants of the Tm-2-2 gene conferring resistance against at least ToBRFV, and preferably also against at least one of TMV, ToMV and ToMMV, comprising:
Expressing transiently or constitutively in a surrogate plant host, preferably in a Solanaceae plant, even more preferably in a Nicotiana species or a Capsicum species, the mutated Tm-2-2 gene to be tested in presence of the movement protein (MP) of ToBRFV, and
Detecting an interaction between the protein expressed from the mutated gene and the ToBRFV MP protein, and preferably detecting hypersensitive response.

Preferred Nicotiana and Capsicum species are Nicotiana benthamiana, Nicotiana tabacum and Capsicum annuum. This method is illustrated in the experimental section of the application, in Nicotiana benthamiana and Nicotiana tabacum plants. In case resistance against some or all of TMV, ToMV and ToMMV, is also needed, the method can be simultaneously carried out in replacing the ToBRFV MP by the MP of these viruses. Preferably, the mutated Tm-2-2 gene or mutant of the Tm-2-2 gene is as defined in connection with the preceding aspects of the invention, namely with at least one of the C848R, N822C, N822F, N822M, N822Y, N822W, S825H, S825K and S825T substitutions, and with Y, F or W at the position 767, and potentially also with L at the position 655. By this method, further mutations of the Tm-2-2 gene can be easily and rapidly tested. As already stressed, detection of hypersensitive response in this assay is a surrogate for detection of resistance against said tobamoviruses in tomato comprising the mutated Tm-2-2 gene to be tested.

Similarly, in order to identify, detect or select mutants of the TM-2-2 protein conferring resistance against ToBRFV, the method is carried out by transiently or constitutively expressing a nucleotide sequence encoding the mutant of the TM-2-2 protein to be tested.

### Sequences:

SEQ ID No:1: nucleotide sequence of tm2.
SEQ ID No:2: nucleotide sequence of Tm-2.
SEQ ID No:3: nucleotide sequence of Tm-2-2.
SEQ ID No:4: nucleotide sequence of Tm2-14-25.
SEQ ID No:5: nucleotide sequence of Tm2-467.
SEQ ID No:6: amino acid sequence of protein encoded by tm2.
SEQ ID No:7: amino acid sequence of TM-2 encoded by Tm-2.
SEQ ID No:8: amino acid sequence of TM-2-2 encoded by Tm-2-2.
SEQ ID No:9: amino acid sequence of TM2-14-25 encoded by Tm2-14-25.
SEQ ID No:10: amino acid sequence of TM2-467 encoded by Tm2-467.
SEQ ID No:11: amino acid sequence of LRR domain of TM-2-2
SEQ ID No:12: nucleotide sequence encoding the LRR domain of TM-2-2
SEQ ID No:13: amino acid sequence of Movement Protein of TMV.
SEQ ID No:14: amino acid sequence of Movement Protein of ToMV.
SEQ ID No:15: amino acid sequence of Movement Protein of ToBRFV.
SEQ ID No:16: amino acid sequence of Movement Protein of ToMMV.
SEQ ID No:17: amino acid sequence of TM2-4.
SEQ ID No:18: amino acid sequence of TM2-5.
SEQ ID No:19: amino acid sequence of TM2-825H.
SEQ ID No:20: amino acid sequence of TM2-825K.
SEQ ID No:21: amino acid sequence of TM2-825T.
SEQ ID No:22: amino acid sequence of TM2-822C.
SEQ ID No:23: amino acid sequence of TM2-822-F.
SEQ ID No:24: amino acid sequence of TM2-822-M.
SEQ ID No:25: amino acid sequence of TM2-822-Y.
SEQ ID No:26: amino acid sequence of TM2-822-W.
SEQ ID No:27: nucleotide sequence of TM2-4.
SEQ ID No:28: nucleotide sequence of TM2-5.
SEQ ID No:29: nucleotide sequence of TM2-825H.
SEQ ID No:30: nucleotide sequence of TM2-825K.
SEQ ID No:31: nucleotide sequence of TM2-825T.
SEQ ID No:32: nucleotide sequence of TM2-822C.
SEQ ID No:33: nucleotide sequence of TM2-822-F.
SEQ ID No:34: nucleotide sequence of TM2-822-M.
SEQ ID No:35: nucleotide sequence of TM2-822-Y.
SEQ ID No:36: nucleotide sequence of TM2-822-W.
SEQ ID No:37: nucleotide sequence of primer npt2F.
SEQ ID No:38: nucleotide sequence of primer npt2R.
SEQ ID No:39: nucleotide sequence of primer tm2-2-F2.
SEQ ID No:40: nucleotide sequence of primer thsp-R.
SEQ ID No:41: nucleotide sequence of binary plasmid pJL469.
SEQ ID No:42: nucleotide sequence of binary plasmid pJL470.
SEQ ID No:43: nucleotide sequence of binary plasmid pJL471.
SEQ ID No:44: nucleotide sequence of primer LM_TBRFV-1-F.
SEQ ID No:45: nucleotide sequence of primer LM_TBRFV-1-R.
SEQ ID No:44: nucleotide sequence of probe LM_TBRFV-1-probe.

### LEGEND OF THE FIGURES

FIG. 1: Transient expression in N. benthamiana of different TM-2-2 variants in presence or absence of movement proteins of TMV or ToBRFV. Photo taken about 5 days post infiltration.

FIG. 2: Transient expression in N. benthamiana of different TM-2-2 variants in presence or absence of movement proteins of TMV or ToBRFV. Leaves of 2 different ages of N. benthamiana plants were infiltrated. About 50 day old plant (Left) or 43 day old plant (Right) were infiltrated with Agrobacterium cultures to transiently express different TM-2-2 protein variants in the presence or absence of movement proteins of TMV or ToBRFV. Photographs taken approximately 5 days after infiltration.

FIG. 3: plasmids for transformation of tomato

FIG. 3A: binary plasmids pJL469 (SEQ ID NO:41)

FIG. 3B: binary plasmids pJL470 (SEQ ID NO:42),

FIG. 3C: binary plasmids pJL471 (SEQ ID NO:43).

FIG. 4: Alignment of proteins of genes Tm2-2, Tm-2-14-25 and Tm2-467.

FIG. 5: Transient expression in N. benthamiana and N. tabacum of different TM-2-2 variants in presence or absence of movement proteins of ToBRFV. TM22 848R variants with different amino acids at AA 767 were transiently expressed in presence (+MP) of absence of movement protein of ToBRFV. The various amino acids at position 767 are labeled on the Figure using the standard single amino acid code to identify variant. Photo taken 2 days after agroinfiltration.

FIG. 5A: N. benthamiana

FIG. 5B: N. tabacum

### EXAMPLES

The TM-2-2 protein (product of the Tm-2-2 gene) is a nucleotide binding leucine rich repeat protein (NLR) and confers resistance to TMV and ToMV by binding the movement protein (MP) produced from either virus, and signaling an effective immune response against the invading virus. The result of binding between TM-2-2 and tobamovirus MP can be observed as a hypersensitive response (tissue necrosis) in a transient expression assay in N. benthamiana.

The NLR proteins TM-2 and TM-2-2 differ by only 4 amino acids (Lanfermeijer et al. 2005). These differences are associated with different spectrums of resistance. For example the TM-2-2 variant can confer resistance to a broader range of TMV and ToMV isolates than TM-2 (Lanfermeijer et al. 2005; Lanfermeirjer 2004). One amino acid change in particular, amino acid 767 in the LRR domain, was demonstrated to be responsible for the more durable and broader host range of resistance in the TM-2-2 protein (Kobayashi et al. 2011). TM-2 and TM-2-2 proteins however provide no resistance against ToBRFV.

The present inventors have thus hypothesized that mutations in the TM-2 and TM-2-2 proteins could retain the capacity to recognize ToMV and TMV MP while conferring the capacity to also recognize ToBRFV MP.

Using the transient expression assay in N. benthamiana in order to test the capacity to recognize tobamovirus MP, the inventors have been able to test efficiently a high number of variants. They have unexpectedly isolated variants of the TM-2-2 protein which efficiently recognize the movement protein (MP) of ToBRFV, triggering a hypersensitive response when co-expressed along with the ToBRFV MP (effector). Furthermore these variants also bind and respond to the ToMV and TMV MP.

The DNA sequences of these variants have been obtained, as well as sequences of further variants also recognizing ToBRFV MP in addition to TMV and ToMV MP (examples 2, 3, 4 and 5).

By TILLING, plants comprising TM-2-2 mutants as described can be obtained (example 6), as well as by Agrobacterium transformation (example 7).

### Example 1: Material and Methods

1.1. Sequences of the Tm-2 Gene Alleles and of the MP Proteins Referred to in this Section:

TABLE 1
List of some tm2 gene alleles.

| Gene | | | |
|---|---|---|---|
| Name | SEQ # | Protein | Phenotype induced and comments |
| tm2 | AF536199 | SEQ ID No: 6 | TMV-U1 (S), ToMV-GeRo (S); ToBRFV (S); |
| | SEQ ID No: 1 | | No HR with ToBRFV MP in N. benthamiana |
| | | | test |
| Tm-2 | AF536200 | TM-2 | TMV-U1 (R); ToMV-GeRo (S); ToBRFV (S); |
| | SEQ ID No: 2 | SEQ ID No: 7 | No HR with ToBRFV MP in N. benthamiana |
| | Plasmid pJL398 | | test |
| Tm-2² | AF536201 | TM-2-2 | TMV_U1 (R); ToMV-GeRo (R); ToBRFV (S); |
| | SEQ ID No: 3 | SEQ ID No: 8 | No HR with ToBRFV MP in N. benthamiana |
| | Plasmid pJL366 | | test |
| Tm-2¹⁴⁻²⁵ | SEQ ID No: 4 | SEQ ID No: 9 | HR with TMV, ToMV and ToBRFV MP in N. |
| | | | benthamiana test |
| Tm2⁻⁴⁶⁷ | SEQ ID No: 5 | SEQ ID No: 10 | HR with ToBRFV MP in N. benthamiana test |
| | Plasmid pJL467 | | |
| Tm2-4 | SEQ ID No: 27 | TM2-4 | HR with ToBRFV MP in N. benthamiana test |
| | Plasmid 517_6 | SEQ ID No: 17 | |
| Tm2-5 | SEQ ID No: 28 | TM2-5 | HR with ToBRFV MP in N. benthamiana test |
| | Plasmid | SEQ ID No: 18 | |
| | pJL_517_7 | | |
| Tm2-825H | SEQ ID No: 29 | TM2-825H | HR with ToBRFV MP in N. benthamiana test |
| | Plasmid 564_25 | SEQ ID No: 19 | |
| Tm2-825K | SEQ ID No: 30 | TM2-825K | HR with ToBRFV MP in N. benthamiana test |
| | Plasmid 565_10 | SEQ ID No: 20 | |
| Tm2-825T | SEQ ID No: 31 | TM2-825T | HR with ToBRFV MP in N. benthamiana test |
| | Plasmid 565_5 | SEQ ID No: 21 | |
| Tm2-822C | SEQ ID No: 32 | TM2-822C | HR with ToBRFV MP in N. benthamiana test |
| | Plasmid 607_5 | SEQ ID No: 22 | |
| Tm2-822F | SEQ ID No: 33 | TM2-822F | HR with ToBRFV MP in N. benthamiana test |
| | Plasmid 607_11 | SEQ ID No: 23 | |
| Tm2-822M | SEQ ID No: 34 | TM2-822M | HR with ToBRFV MP in N. benthamiana test |
| | Plasmid 608_18 | SEQ ID No: 24 | |
| Tm2-822Y | SEQ ID No: 35 | TM2-822Y | HR with ToBRFV MP in N. benthamiana test |
| | Plasmid 607_12 | SEQ ID No: 25 | |
| Tm2-822W | SEQ ID No: 36 | TM2-822W | HR with ToBRFV MP in N. benthamiana test |
| | Plasmid 606_1 | SEQ ID No: 26 | |

TMV-U1 = Tobacco Mosaic virus U1 strain
ToMV = Tomato Mosaic Virus GeRo Strain
ToBRFV = Tomato Brown Rugose Fruit virus
HR = hypersensitive response
R = resistant; S = Susceptible

TABLE 2
Amino acid sequences of selected Tobamovirus
MPs referred to in the examples.

| MP Source | AA seq | Plasmid name |
|---|---|---|
| TMV (AF546184.1 Flavum strain) | SEQ ID No: 13 | pJL 380 |
| ToMV (X02144.1 OM strain) | SEQ ID No: 14 | pJL 481 |
| ToBRFV (MS549394.1 Ca1A isolate) | SEQ ID No: 15 | pJL 379 |
| ToMMV (KX898033) | SEQ ID No: 16 | |

### 1.2. Mutagenesis:

To find TM-2-2 protein variants that trigger HR in the presence of ToBRFV MP (MP_Rugose in the following), two different approaches were deployed, namely random and site-directed mutagenesis, each designed to create variation in the LRR domain of TM-2-2.

Using the Takara Diversify PCR Random Mutagenesis Kit and PCR conditions that generated, on average, 4 changes per 1000 basepairs, the last around 750 nucleotides of the TM-2-2 gene were amplified by the PCR. The resulting PCR product was cloned into a Tm-2-2 gene expression plasmid, replacing the wt 3′˜750 nts of the Tm-2-2 gene.

When it is desirable to introduce variation at specific locations in the Tm-2-2 gene site directed mutagenesis can be used. This is accomplished by the synthesis of portions of the Tm-2-2 gene with sequence variation at specific codons of interest. For example a synthetic oligonucleotide that can be used as a primer in the PCR can be designed for amplification of part of the Tm-2-2 gene, yet still designed to have one or more non-wild type nucleotides at specific locations. Following the PCR with such an oligonucleotide primer, the PCR product can be cloned into the appropriate location of the Tm-2-2 gene. In this manner it is possible to introduce nucleotide diversity at specific locations in a gene.

1.3. Transient Expression in N. benthamiana and Evaluation of Necrosis:

Transient expression in N. benthamiana and N. tabacum was carried out essentially as described in Ma et al, and in Kobayashi et al.

The products of the ligation reaction disclosed in example 1.2 were transformed into Agrobacterium tumefaciens (GV3101) and plated on LB plates with 50 ug/ml Kanamycin and 25 ug/ml Gentamycin to select for transformed Agrobacterium.

Approximately 860 different Agrobacterium colonies were selected from the transformation. Each colony grown in liquid culture and used to prepare an Agrobacterium suspension for agroinfiltration using standard procedures commonly used in plant biology (Tomita et al. 2019). Prior to infiltration into N. benthamiana leaves, cultures were mixed 1:1 with a suspension of Agrobacterium that had been transformed with the tobamovirus MP expression plasmid, for example the ToBRFV MP expression plasmid pJL 379.

Infiltrated plants were kept under either LED or fluorescent lights (18 hr light, 6 hr dark) at between 22 and 25 C. With an error rate of approximately 4 changes per kb it was estimated that approximately 2500 nucleotides changes were sampled (750 bp/clone×860 clones×4 errors/1000 bp=˜2580 nt changes). In initial characterization of this library by sequencing the inventors estimated about 75% of the nucleotide changes would result in amino acid changes. If 75% of all the nucleotide mutations in the clones screened generated amino acid changes this would be approximately 1900 AA changes to the TM-2-2 protein were screened (0.75×2580=1935)

Approximately 6 days post infiltration infiltrated leaves were observed and the degree of necrosis (Hypersensitive Response, HR) in infiltrated zones were measured. Typically a scale from 0 to 4 was used to estimate the percentage of infiltrated zone showing necrosis. Details of the scale are as follows: 0=0% HR, 1=˜25% HR, 2=˜50% HR, 3=˜75% HR, 4=100% HR. As a control, plants were also infiltrated with a 1:1 mix of Agrobacterium carrying the wt Tm-2-2 expression plasmid (pJL 366) and Rugose MP expression plasmid (pJL 379).

### 1.4. Protocol for Evaluation of Tobamovirus Resistance:

Several Tobamovirus isolates were used to perform Bioassay: ToBRFV isolates (inter alia Jordan_2015), ToMV, TMV or other tobamovirus.

Virus isolates are maintained by frozen storage of infectious juice coming from 14 days old infected tomato leaves grinded in water (inoculum proportion: 1 g leaves for 4 ml of water). Bioassay are carried out by sap-inoculation of tomato plantlets at two-leaf stage (i.e. 14-16 days after seeding) by rubbing the cotyledons with the index finger. At least 18 plantlets (separated in 2 or 3 repetitions) per tomato lines/accessions/genotypes were tested for tobamovirus resistance.

Phenotypic evaluation of plant is carried out by plant by plant scoring, without contacting the plants. The presence of local lesions on the inoculated organs is carried out between 7 and 10 DPI. If at least one plant per genotype exhibits local lesions on inoculated organs, the genotype is considered as potentially interesting. The tobamoviruses indeed, do not generally trigger necrosis on susceptible plants.

Systemic symptoms evaluations are carried out at 14, 21 and 28 DPI, the last evaluation being optional. Symptoms are visually assessed according the following scale: 9: No visible symptoms/7: small phenotypic difference but not clearly attributable to a disease symptom/5: mild symptoms (mosaic and/or light vein banding)/3: strong symptoms (strong mosaic and/or pronounced vein banding and/or small leaves deformation)/1: very strong symptoms (leaves deformation and/or mosaic and/or highly pronounced vein banding).

After 14 and/or 28 days of test, the plants without symptoms are tested by ELISA and/or quantitative PCR to evaluate the presence of tobamovirus in plant.

### 1.5. TILLING (Targeting Induced Local Lesions IN Genomes):

Tilling method is applied according to the usual protocols.

All DNA reactive chemical agents (mutagens) can be used for inducing lesions in the DNA and are not limited to EMS.

Physical DNA reactive agents (mutagens) can also be used, such as:
Ionizing radiations (X-Ray, gamma rays, alpha particles . . . ) heavy-ion beam irradiation
Ultraviolet radiations
Radioactive decay

The physical or chemical mutagen is applied on M0 seed. The M1 plants, heterozygous for all mutations introduced by the mutagen are then selfed, giving rise to the seeds M2. A portion of these seeds are stored. A sampling is then conducted on at least 8 plants M2 for sequencing. The theoretical ratios are ½ of the plants will be heterozygous for a given mutation, ¼ will be homozygous for the mutation and ¼ will be homozygous for the absence of said mutation.

Alternatively, the screening for suitable mutations can also be conducted on M1 plants.

In the present case, insofar as the expected mutation in the Tm-2-2 gene is highly specific, it is important to test a very large population of mutants.

### Example 2: Identification of a Tm2 Gene Allele that Recognizes ToBRFV MP

### BACKGROUND

Tomato Brown Rugose Fruit Virus (ToBRFV) is a serious pathogen of tomatoes and peppers. It is related to other tobamoviruses such as Tobacco Mosaic Virus (TMV) and Tomato Mosaic Virus (ToMV).

Genetic Resistance against TMV and ToMV maps to the Tm-2-2 gene. The TM-2-2 protein, product of the Tm-2 gene, is a resistance (R) protein of the nucleotide binding leucine rich repeat (NLR) class, also known as NBS-LRR class. There are two commonly use alleles of the Tm-2 gene in commercial tomato germplasm, namely Tm-2 and Tm-2-2. The different alleles confer resistance to different tobamoviruses (see Table 1). The Tm-2-2 allele in particular is widely deployed in commercial tomato germplasm because it confers durable genetic resistance to two tobamoviruses (TMV and ToMV) which are significant disease threats to tomato. However ToBRFV can infect plants that carry the Tm-2 or Tm-2-2 genes and there are no identified Tm-2 alleles that confer resistance to ToBRFV. Moreover, there are no known resistance genes that provide effective resistance simultaneously against ToBRFV, TMV and ToMV.

The TM-2-2 protein confers resistance to TMV and ToMV by binding the movement protein (MP) produced from either virus, and signaling an effective immune response against the invading virus by triggering a hypersensitive response (HR) in tomato, resulting in cell death. The result of binding between TM-2-2 and tobamovirus MP, namely the HR response, can be also observed by transiently expressing TM-2-2 protein and either TMV or ToMV MP in leaves of N. benthamiana plants, following the protocol disclosed in Kobayashi et al, 2011.

Transient expression of TM-2-2 and either TMV or ToMV MP in N. benthamiana leaves (by the Agroinfiltration technique) indeed results in a hypersensitive reaction and extensive tissue necrosis in just a few days. In contrast transient expression of TM-2-2 and ToBRFV MP (MP_Rugose) in N. benthamiana leaves usually results in no, or occasionally very mild, necrosis.

The HR response assay in N. benthamiana has previously been used to identify specific amino acids in the NLR protein which are critical for recognizing tobamovirus MPs (Kobayashi et al, 2011). There is a perfect correlation between the ability of a TM2 NLR protein to trigger a robust HR response in N. benthamiana leaves (in the presence of a tobamovirus MP) and virus resistance in tomato. Moreover, transient expression of proteins by Agroinfiltration is a well established technique in plant biology.

In view of this perfect correlation, this assay in N. benthamiana can be used as a surrogate for resistance in tomato.

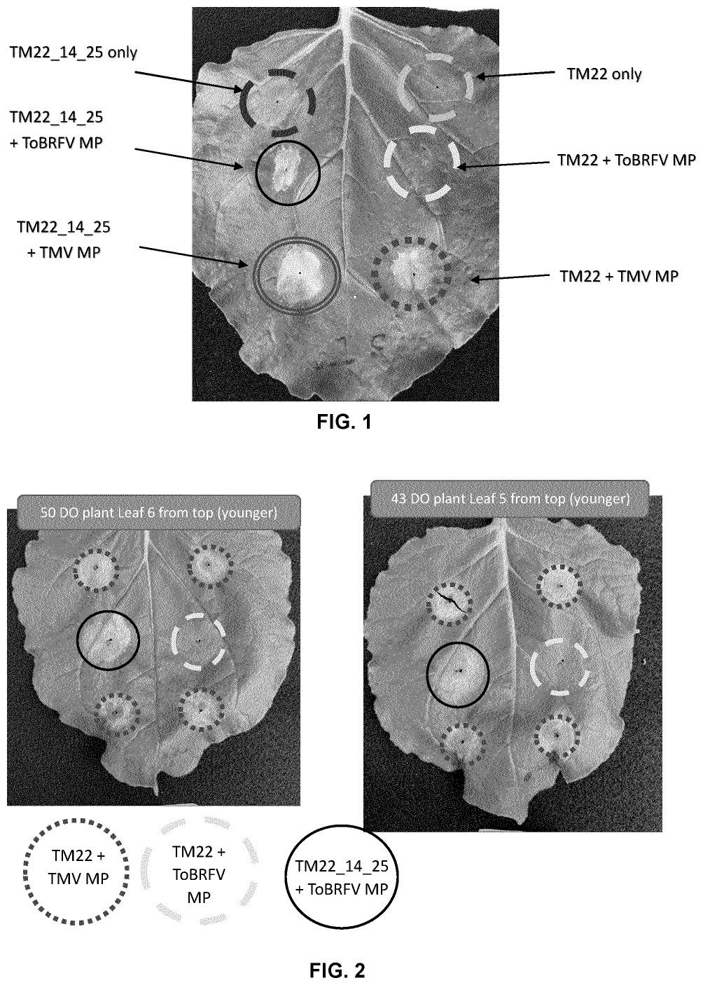

However, whereas the prior uses of this assay or method aimed at identifying the specific amino acids in the NLR protein which are critical for recognizing tobamovirus MPs, by detecting the mutations giving rise to loss of function, the inventors have for the first time used this method to test mutants likely to provide a gain of function, i.e. the capacity to recognize further tobamovirus MPs. The inventors have first generated a library of mutants of the Tm-2-2 gene using error prone PCR methods. This library of variants was then transiently expressed along with the ToBRFV MP in leaves of N. benthamiana plants (see Material and Methods in examples 1.2 and 1.3). After screening more than 860 transformed agrobacterium cultures, corresponding in average to 2.25 amino-acid modifications per variant, the inventors identified one colony (LP 14-25) showing a repeatable and noticeably increased HR response (as compared to the control) when co-expressed with ToBRFV MP. The inventors have moreover checked that this variant, triggering a robust HR response in the presence of ToBRFV MP, has not lost its ability to trigger also a robust HR response in the presence of TMV or ToMV MP (See FIGS. 1 and 2 and table 3).

TABLE 3
HR response of various TM2 protein variants to
tobamovirus movement proteins.

| | TMV_U1 MP | ToBRFV MP |
|---|---|---|
| Protein | SEQ ID No: 13 | SEQ ID No: 15 |
| tm2 | − | − |
| Tm-2-2 | + | − |
| Tm2-14-25 | + | + |
| Tm2-467 | + | + |

− = no HR response when proteins are co-expressed in N. benthamiana leaves.
+ = strong HR response when proteins are co-expressed in N. benthamiana leaves.

Detailed analysis of this variant revealed that it was different from the TM-2-2 protein at 2 amino acids (F655L and C848R). This new variant is called Tm2-14-25 (see Table 1).

Through additional analysis, using standard molecular techniques, including site-directed mutagenesis, the inventors mapped the ability to recognize ToBRFV MP to a single amino acid change (C848R). The inventors identify this new Tm-2 allele as Tm2-467 (see Table 1 and Table 4).

This experiment revealed that C848R change to TM-2-2 was both involved and sufficient for detection and response to MP-Rugose (ToBRFV MP).

TABLE 4
reports the HR response of different variants:

| Construct | AA changes to | HR when expressed |
|---|---|---|
| Name | TM-2-2 | with MP Rugose |
| LP_14-25 | F655L, C848R | 4 |
| pJL 466 | F655L | <1 |
| pJL 467 | C848R | 4 |
| pJL 366 | None (wt TM-2-2) | <1 |

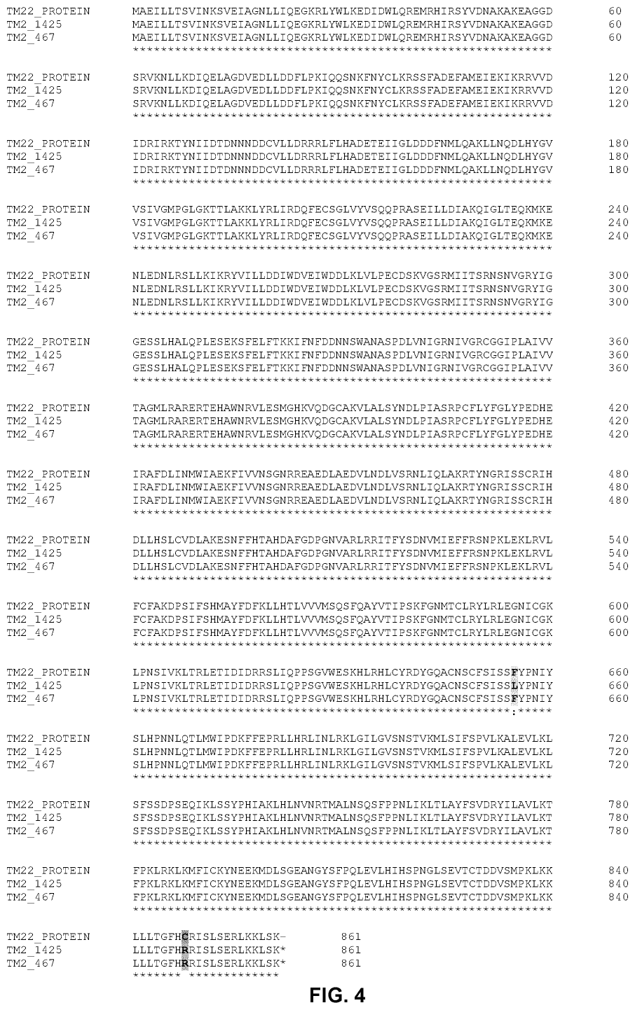

An alignment of the proteins TM2-2, TM2-14-25 and TM2-467 (protein products of the Tm-2-2, Tm2-14-25 and Tm2-467 genes, respectively) is presented in FIG. 4.

Since both TM2-14-25 and TM2-467 proteins recognize ToBRFV MP it is apparent that the amino acid change they share is responsible for the ability to robustly recognize ToBRFV MP (as compared to TM-2-2 protein). It is also observed that the protein can have additional mutations (such as the change unique to TM2-14-25) and still recognizes ToBRFV MP. Both TM2-14-25 and TM2-467 proteins still recognize TMV and ToMV MPs.

Either TM2_14-25 or TM2-467 protein sequences can confer improved resistance to ToBRFV in tomato, while simultaneously conferring resistance against TMV and ToMV.

### Example 3: Additional Mutants

As detailed in the preceding example, the inventors have shown that a single amino acid change in the TM-2-2 protein is sufficient for triggering a robust HR response in the presence of ToBRFV MP, without loss of resistance against TMV and ToMV.

In order to better characterize this mutation, and additional mutations likely to improve the resistance, other amino acid variations were created, using a site-directed approach. Selected codons in the LRR domain were changed to encode for non-wild type amino acids. Functional screening of the protein variants was performed by agroinfiltration and transient expression in N. benthamiana in the presence of MP_rugose (as described above). In some cases, selected amino acid changes were screened in a TM-2-2 protein background that also had the 848R change discussed in example 2. Results of some of the screens are shown in Tables 5-11.

### Position 848

TABLE 5
Tests for mutants at position 848

| Construct | AA at | HR when expressed |
|---|---|---|
| Name | 848 | with MP Rugose |
| pJL 366 | C (wt) | <1 |
| pJL 468_A | A | <1 |
| pJL 468_D | D | <1 |
| pJL 468_E | E | <1 |
| pJL 468_H | H | <1 |
| pJL 468_K | K | ~1 |
| pJL 468_L | L | <1 |
| pJL 468_M | M | <1 |
| pJL 468_N | N | <1 |
| pJL 468_P | P | <1 |
| pJL 468_Q | Q | <1 |
| pJL 468_R | R | 4 |
| pJL 468_S | S | ~1 |
| pJL 468_T | T | ~1 |

Conclusion: None of the amino acid variations at position 848 creates a TM-2-2 variant that elicits a similar let alone a stronger hypersensitive response in the presence of MP_Rugose than C848R.

### Position 857

The impact of AA variation at position 857 was also tested, this position being near position 848 and in the vicinity of amino acid 848 in the 3D structure of the TM-2-2 protein. The results are presented in table 6.

TABLE 6
Tests for mutants at position 857,
with or without C848R variation.

| Construct | | | HR to MP |
|---|---|---|---|
| Name | AA 848 | AA 857 | Rugose |
| pJL 366 | C (wt) | K (wt) | <1 |
| pLP 14-25 | R | K | 3 |
| pJL 480Q | R | Q | 3 |
| pJL 480E | R | E | <1 |
| pJL 480T | R | T | <1 |
| pJL 480R | R | R | <1 |
| pJL 480I | R | I | <1 |

Conclusion: several AA changes at position 857, with AA 848 as R, reduce the HR response to MP Rugose. Other AA changes near 848R can however preserve the response to MP Rugose.

Variations at position 857 are thus allowable but to a limited extent. Acceptable variations can be easily tested.

### Position 767

The impact of AA variation at position 767 (only) was also tested (without variation at position 848). The amino acid at this position has been demonstrated by Kobayasi et al as being decisive for the differences between TM-2 and TM-2-2 resistances. The results are presented in table 7.

TABLE 7

| Construct Name | AA 767 | HR to MP Rugose |
|---|---|---|
| pJL 366 | Y (wt) | <1 |
| L11_10 | I | 0 |
| L11_11 | C | 0 |
| L11_15 | L | 0 |
| L11_16 | G | 0 |
| L11_19 | N | 0 |
| L11_20 | V | 0 |
| L11_1 | R | <1 |

Conclusion: Of 7 different AA variants at position 767 tested, none improved the ability to detect and respond to MP Rugose, in the absence of theC848R mutation.

### Position 769

The impact of AA variation at position 767 (only) was also tested (without variation at position 848). The results are presented in table 8.

TABLE 8

| Construct Name | AA 769 | HR to MP Rugose |
|---|---|---|
| pJL 366 | S (wt) | <1 |
| L12_11 | G | <1 |
| L12_12 | R | <1 |
| L12_4 | F | <1 |
| L12_16 | E | <1 |
| L12_3 | V | <1 |
| L12_A | A | 0 |

Conclusion: Testing TM22 variants with 6 different (non wt) AA at position 769 did not reveal any variants that are better at binding and responding to MP Rugose, than the C848R variant.

### Position 767 in the TM-2-2 848R Background

The impact of AA variation at position 767, in addition to the C848R variation, was also tested. The results are presented in table 9.

TABLE 9

| Construct Name | AA 767 | AA 769 | AA 848 | HR to MP Rugose |
|---|---|---|---|---|
| pJL 511_1 | S | A | R | 0 |
| pJL 511_2 | S | S (wt) | R | 0 |
| pJL 511_3 | Y | A | R | 4 |
| pJL 511_5 | Y (wt) | S (wt) | R | 4 |
| pJL 511_8 | D | A | R | 0 |
| pJL 511_12 | D | S (wt) | R | 0 |

Conclusion: Detection of MP Rugose is dependent upon the AAs at both positions 767 and 848. The TM-2-2 variant with AA 848 as R and 767 as Y can detect and respond to MP_Rugose. However changing AA 767 to S or D dramatically reduces the ability of the protein to respond to MP_Rugose. This indicates that both residues 767 and 848 are important for binding MP_Rugose.

Further investigations by the inventors have demonstrated that position 767 can however be substituted by W and F without significantly reducing the ability of the protein to respond to MP_Rugose, and even enhancing this ability (see example 4).

### Position 822

The impact of AA variation at position 822 was also tested, in the context of the C848R variation, this position 822 being proposed to be in the vicinity of amino acid 848 in the 3D structure of the TM-2-2 protein. The results are presented in table 10.

TABLE 10

| Construct Name | AA 822 | AA 848 | HR to MP Rugose |
|---|---|---|---|
| pJL 366 | N (wt) | C (wt) | <1 |
| pLP 14-25 | N | R | 4 |
| pJL 476_S | S | R | 4 |
| pJL 476_I | I | R | 0 |
| pJL 476_F | F | R | 2 |
| pJL 476_C | C | R | 2 |
| pJL 476_T | T | R | <1 |
| pJL 477_H | H | R | <1 |
| pJL 477_D | D | R | <1 |
| pJL 476_K | K | R | 0 |
| pJL 476_R | R | R | 0 |

Conclusions: Various amino acids at position 822, can reduce ability of TM22 848R to bind and respond to MP_rugose but other variations are allowable. Amino acids N and S are preferred at position 822 when 848 is R.

Further investigations by the inventors have demonstrated that position 822 can however be substituted by C, F, M, Y and W and provide the ability of the protein to respond to MP_Rugose even in the absence of the C848R mutation (see example 5).

In summary, in the process of identifying this mutant and further mutants comprising mutations allowing this gain of function, more than 1000 different variants were screened, using a combination of site directed and random mutagenesis. It is to be noted that some members of the libraries of mutants obtained by random mutagenesis were screened in HR assay without being sequenced.

Table 11 below details the different variants which were screened for response to ToBRFV MP, with mention of the tested mutation if known.

TABLE 11
Overview of variants of TM22
screened for response to MP_Rugose

| | | | | Approx # | |
|---|---|---|---|---|---|
| | | AA | | screened | |
| LINE | Library | position | | before | specific |
| ID | type | targeted | Strategy | sequencing | AA tested |
| 1 | site | 767 | Degenerate | | YICLGNVRS |
| | directed | | codons/synthetic oligos | | D |
| 2 | site | 769 | Degenerate | | SGRFEVA |
| | directed | | codons/synthetic oligos | | |
| 3 | random | 611-861 | error prone pcr | 860 | unknown |
| 4 | site | 822 | Degenerate | | NSIFCTYHD |
| | directed | | codons/synthetic oligos | | KR |
| 5 | site | 825 | Degenerate | | AITPCFYNR |
| | directed | | codons/synthetic oligos | | S |
| 6 | site | 827 | Degenerate | 96 | VGLQHFSM |
| | directed | | codons/synthetic oligos | | |
| 7 | site | 848 | Degenerate | | ADEHKLMNP |
| | directed | | codons/synthetic oligos | | QRST |
| 8 | site | 851 | Degenerate | | ACEFGHKLN |
| | directed | | codons/synthetic oligos | | PQRSTVW |
| 9 | site | 857 | Degenerate | | KQETRI |
| | directed | | codons/synthetic oligos | | |
| | | | TOTAL | 956 | 81 |

In Summary, 767Y and 848R in TM-2-2 appear as critical residues for binding and responding to MP_Rugose according to these tests (further modifications appear however to be permissive, see example 4). Other amino acid changes at other locations of the LRR domain can often lead to mild or sometimes significant, decreases in MP_Rugose binding, but not all; suitable amino acid changes can easily be tested by the assay described in the present invention.

The amino acids in the vicinity of amino acid 848 in the 3D structure of TM-2-2 are likely to be less prone to mutations without loss of binding to the ToBRFV.

In other experiments libraries of variants at a single codon were screened. An example of one such experiment, at codon 827, and its results are shown below:

Degenerate codon libraries at codon 827 in TM-2-2 848R gene background

| Library | Degenerate | | # isolates | |
|---|---|---|---|---|
| Name | codon | Possible AA (#) | screened | HR response to MP Rugose |
| Library 1 | HWK | K,N,M,I,Q,H,L,Y,F (9) | 41 | NONE better than TM22 848R |
| Library 2 | KVS | D,E,A,G,S,C,W,Y (7) | 41 | NONE better than TM22 848R |
| Library 3 | MSA | T,R,P,L | 14 | NONE better than TM22 848R |

Degenerate codon libraries screened at codon 827 in TM22 (848C) gene background

| | | | # isolates | |
|---|---|---|---|---|
| Library | Codon | AA possible (#) | screened | HR to MP Rugose |
| Library 4 | HWK | K,N,M,I,Q,H,L,Y,F (9) | 41 | NONE better than TM22 848R |
| Library 5 | KVS | D,E,A,G,S,C,W,Y (7) | 41 | NONE better than TM22 848R |
| Library 6 | MSA | T,R,P,L | 14 | NONE better than TM22 848R |

The results obtained with libraries 1-6, similar to results obtained at other positions, demonstrate that the variation at position 848 is very important and that variations of other codons in the LRR region of TM-2-2 do not produce variants that have an improved recognition and response to MP_rugose. In any event, the mutation at position 848 appears necessary for the MP_rugose recognition.

### Example 4: Further Modification at Position 767 and Validation in Different Nicotiana Species

Further modifications at position 767 of the TM-2-2 protein variant comprising the C848R substitution have be tested in different Nicotiana species.

Specifically, plants (N. benthamiana and N. tabacum) were infiltrated with Agrobacterium cultures containing plasmids with T-DNAs for expression of a TM-2-2 protein variant alone, or co-expression of a TM-2-2 protein variant and MP_Rugose.

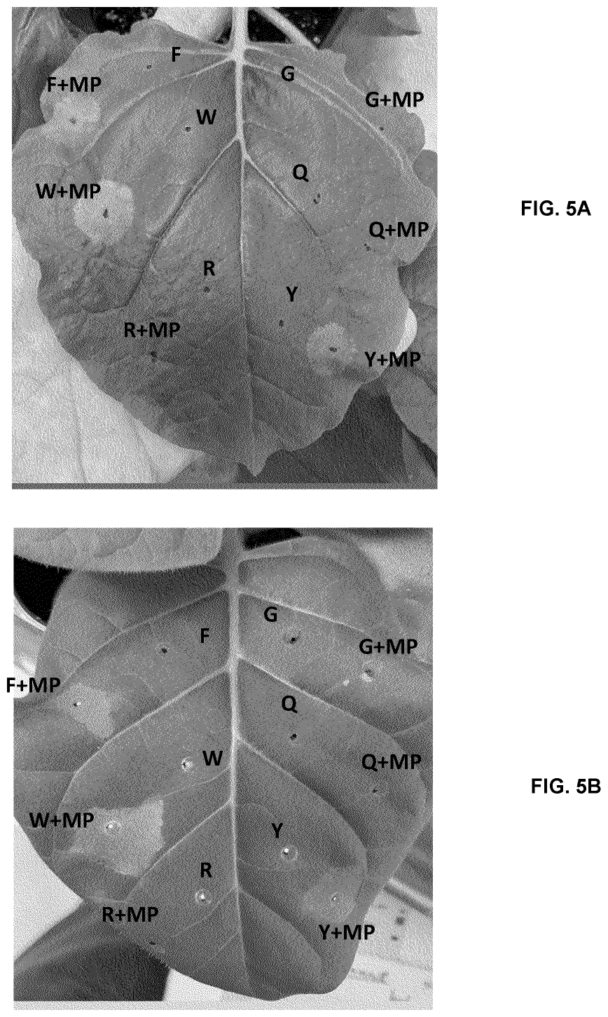

The results are presented on FIG. 5. Letter by spots on leaf denote the amino acid at position 767 in TM-2-2 848R variant background. TM-2-2 variants were expressed alone (Letter only) or co-expressed with Rugose MP (+MP).

Plants have been photographed˜48 hours post infiltration.

Results show that TM-2-2 767Y 848R (TM2-467); 767F 848R (TM2-4), and 767W 848R (TM2-5) all trigger an HR response in presence of MP_Rugose in either N. benthamiana (FIG. 5A) or N. tabacum cv Xanthi (FIG. 5B). The 767W and Y variants appear to be more ‘responsive’ to MP rugose than the 767F variant in this assay. In contrast TM-2-2 variants with R, Q or G amino acids at position 767 did not trigger HR in presence of MP_Rugose.

### Example 5: Identification of Further Tm2 Gene Alleles that Recognize ToBRFV MP

In view of the importance of the C848R mutation, further mutants have been tested wherein amino acids in the vicinity of C848, in the 3D-structure, are mutated. Namely the 20 different amino acids have been tested at position 822 and at position 825 of the TM2-2 protein, in the absence of the C848R mutation.

Plants (N. benthamiana) were infiltrated with Agrobacterium cultures containing plasmids with T-DNAs for expression of a TM-2-2 protein variant alone, or co-expression of a TM-2-2 protein variant and MP_Rugose.

The variants giving rise to an intense hypersensitive response are reported in table 12, with the details of the alleles at positions 767, 822, 825 and 848. All other variants at positions 822 and 825, in combination with C848 (wt) and Y767 (wt), do not give rise to HR to MP Rugose.

TABLE 12
further variants at position 822 and 825 and results of the HR to MP Rugose test

| Name of | Position | Position | Position | Position | HR to MP | Nb of AA changes wrt | Plasmid |
|---|---|---|---|---|---|---|---|
| the allele | 767 | 822 | 825 | 848 | Rugose | TM2-2 (SEQ ID No. 8) | name |
| TM2-825H | Y (wt) | N (wt) | H | C (wt) | ++ | 1 | 564_25 |
| TM2-825K | Y (wt) | N (wt) | K | C (wt) | ++ | 1 | 565_10 |
| TM2-825T | Y (wt) | N (wt) | T | C (wt) | ++ | 1 | 565_5 |
| TM2_822C | Y (wt) | C | S (wt) | C (wt) | ++ | 1 | 607_5 |
| TM2_822F | Y (wt) | F | S (wt) | C (wt) | ++ | 1 | 607_11 |
| TM2_822M | Y (wt) | M | S (wt) | C (wt) | ++ | 1 | 608_18 |
| TM2_822Y | Y (wt) | Y | S (wt) | C (wt) | ++ | 1 | 607_12 |
| TM2_822W | Y (wt) | W | S (wt) | C (wt) | ++ | 1 | 606_1 |

In conclusion, in addition to the C848R mutation of the TM2-2 protein, the N822C, N822F, N822M, N822Y, N822W, S825H, S825K and S825T mutations of the TM2-2 protein are also sufficient for conferring resistance to ToBRFV, as can be deduced from the HR response to MP_Rugose.

### Example 6: Providing Non-Transgenic Plants Carrying the New Variants of Tm2 Recognizing the ToBRFV MP by TILLING (Targeted Induced Local Lesions in Genomes) Strategy

In Mutagenized Population:

A large variant tomato population is created using a physical or chemical mutagen agent, well known to the skilled person, that induced all kind of random mutations in genomic sequence by nucleotide substitution. The parental line used for the population is a line which preferably carries the Tm2² gene at homozygous level. This line is resistant to ToMV, TMV and susceptible to ToBRFV.

A massive screening of the population is done at M1 or M2 step (protocol detailed in example 1.5) to identify variations in the Tm2 gene (Solyc09 g018220) using well known screening methods, which are preferably molecular assay based or sequenced based.

Plants carrying variations in Tm2 gene are selfed for seed production. The following generation (M2 or M3) are genotyped for the targeted variation and heterozygous or homozygous plants (fixation of the variation) are used for phenotyping and for introgression in Elite line by MABC (marker-assisted backcrossing) or other classical breeding method. Several backcrosses are performed to remove other variations in the genetic background.

Plants carrying the variation are phenotyped for ToBRFV and other tobamovirus resistance using the protocol detailed in example 1.4.

### Example 7: Provision of Transgenic Plants Carrying the New Variants of Tm2 Recognizing the ToBRFV MP

Anabelle tomato line is used for Agrobacterium transformation with Tm2-2 variants as identified in the previous sections of the results.

Seeds are surface-sterilized for 20 min in 2% sodium hypochlorite containing 2 drops of Tween20 under agitation then washed 3 times with sterile distilled water.

Seeds are then cultivated in plastic jars with MS medium (M0222, Duchefa) pH 5.9 containing 20 g/l sucrose and 0.8% microagar and placed at 25° C. under light (3000-4000 lux, 16 h photoperiod). Explants are excised from cotyledons of 10 days seedlings. Using a sterile forceps and razorblade, both ends of each cotyledon are removed and cotyledons are then cut into 2 pieces. Explants are placed onto 10 cm diameter Petri dishes containing CC medium (MS medium pH5.9 containing 20 g/l sucrose and 0.8% microagar supplemented with 2 mg/l NAA, 1 mg/l BAP, 160 mg/l glucuronic acid and 40 mg/l acetosyringone) and placed at 25° C. under light (3000-4000 lux, 16 h photoperiod) for one day.

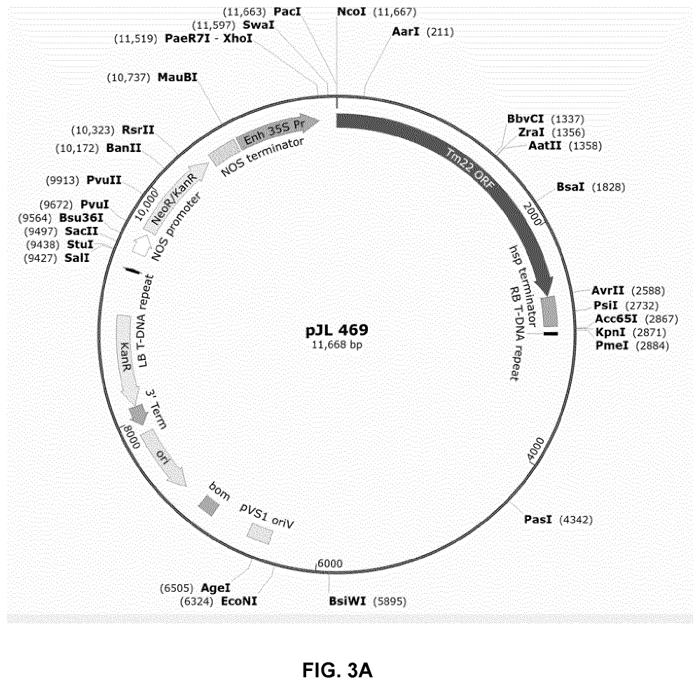

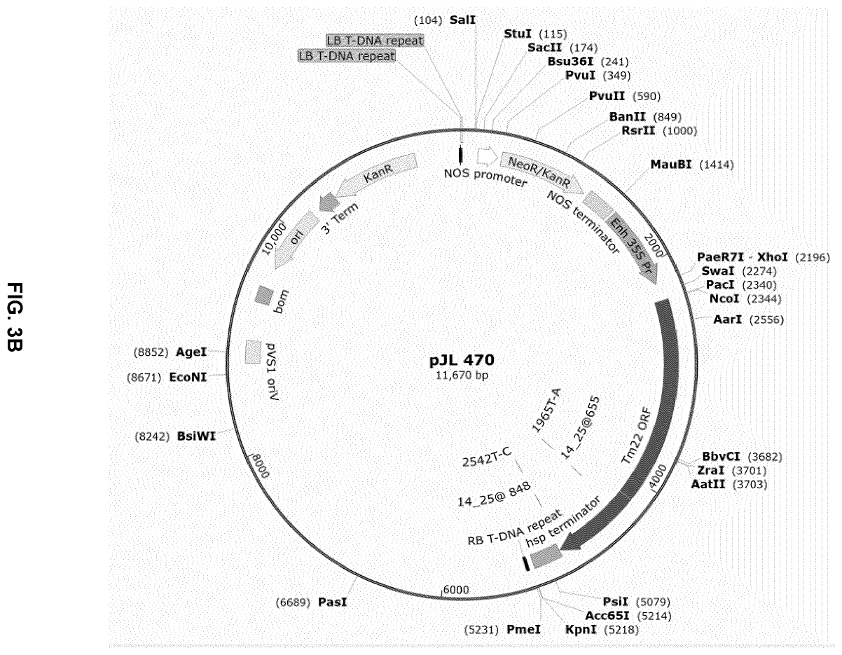

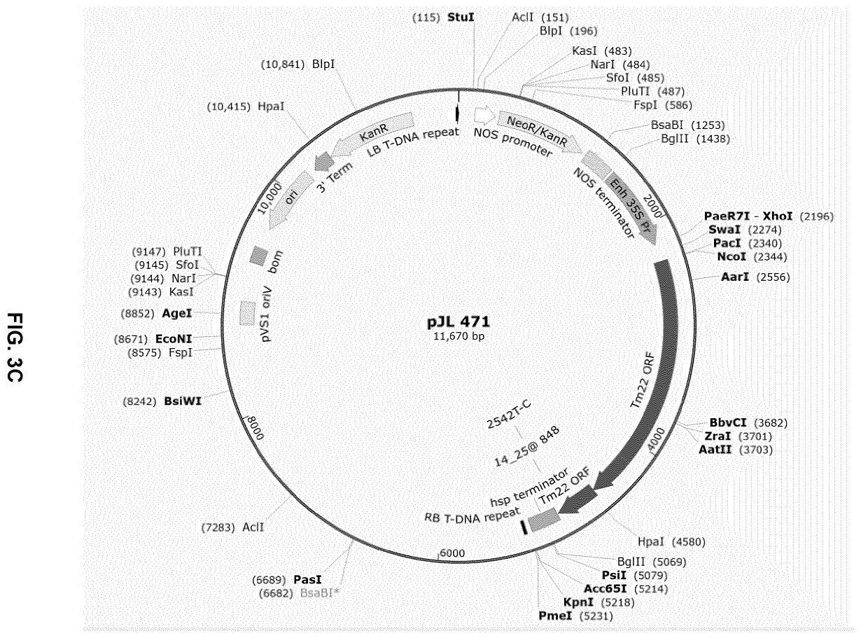

Agrobacterium tumefaciens strains A1224, A1225 and A1250 were obtained by electroporation of the binary plasmids pJL470, pJL471 and pJL469, respectively (see FIGS. 3A, 3B and 3C), into Agrobacterium tumefaciens strain GV3101.

A single colony of Agrobacterium tumefaciens containing T-DNA plasmid was cultivated in 15 ml LB broth containing rifampicin 10 μg/ml and kanamycin 50 μg/ml in a shaker (200 rpm) at 28° C. for 20 h. Bacteria are pelleted by centrifugation of the overnight suspension for 20 mn at 1000 g and resuspended in sterile CC liquid medium (MS medium pH5.9 containing 20 g/l sucrose supplemented with 2 mg/l NAA, 1 mg/l BAP, 160 mg/l glucuronic acid and 40 mg/l acetosyringone) to an OD600 nm of 0.1

In a sterile beaker, cotyledonary explants are soaked in the Agrobacterium suspension for 15 min under slow agitation (100 rpm). With sterile forceps, explants are blotted on a sterile filter paper then transferred to solid CC medium (MS medium pH5.9 containing 20 g/l sucrose and 0.8% microagar supplemented with 2 mg/l NAA, 1 mg/l BAP, 160 mg/l glucuronic acid and 40 mg/l acetosyringone). Plates are placed at 25° C. under light (3000-4000 lux, 16 h photoperiod) for 48 hr

Explants are rinsed two times with 100 ml of liquid MS medium pH 5.9 containing 20 g/l sucrose and supplemented of 100 mg/l amoxicillin 20 mg/l clavulanic acid then blotted onto a sterile paper then transferred to solid selection medium (MS medium containing 20 g/l sucrose and 0.8% microagar supplemented with 1 mg/l zeatin, 100 mg/l amoxicillin 20 mg/l clavulanic acid and 100 mg/l kanamycin) (10 explants/Petri dish). Petri dishes are placed at 25° C. under light (3000-4000 lux, 16 h photoperiod). and medium is refreshed every 2 weeks until shoot regeneration.

Shoots and plantlets are isolated and placed in plastic jars containing rooting medium (MS medium pH5.9 containing 20 g/l sucrose and 0.8% microagar supplemented with 0.5 mg/l IAA, 100 mg/l amoxicillin 20 mg/l clavulanic acid and 100 mg/l kanamycin). Jars are placed at 25° C. under light (3000-4000 lux, 16 h photoperiod).

Well rooted plantlets are transferred to soil. Agar is carefully removed by rinsing roots with water and plants are put in trays with soil and transferred to greenhouse.

Two weeks after acclimatization, plants are sampled and a piece of young leaf is analyzed by flow cytometry to select diploid plants. Genomic DNA is extracted from a young leaf disk and a PCR amplification of nptII gene is performed, using the primers:

Forward primer: npt2F
(SEQ ID NO: 37)
CCTGCCGAGAAAGTATCC
and
Reverse primer: npt2R
(SEQ ID NO: 38)
GCCAACGCTATGTCCTGA

to screen the transformants.

Transformants could be also characterized by PCR amplification with the following primers:

Forward primer: tm2-2-F2
(SEQ ID NO: 39)
TTCCTCCAAATCTCATCAAGC,
and
Reverse primer: thsp-R
(SEQ ID NO: 40)
CAACAAGCCAAGAgAAAACACA.

The plants obtained by this protocol have been tested in order to confirm the resistance to ToBRFV, as well as resistance to TMV, ToMV and ToMMV.

Molecular quantification of ToBRFV sequence in infected plants has also been carried out by quantitative PCR (qPCR) with TaqMan probes, to also confirm the reduction of virus replication.

The protocol for the molecular quantification of ToBRFV by qPCR is as follows:

The young wrapper leaf at head grown are sampled (3 to 4 leaves per plant). The leaves are ground in liquid nitrogen and an aliquot of 100 mg is kept for RNA extraction. For each sample, 100 mg of grounded leaves are used for RNA extraction

RNA extraction is performed using the «Maxwell® 16 LEV Plant RNA Kit» from Promega® and the Maxwell® extraction robot (Promega®). Extracted RNA are stored at −20° C.

qPCR for virus quantification is performed using the TaqMan universal Master Mix (ThermoFisher Scientific®) with Kit Gotaq Probe OneStep RTqPCR system A6120 Promega, following the manufacturer instructions.

Primers and probes used are disclosed in the table below:

| primer | Sequence | SEQ ID NO |
|---|---|---|
| LM_TBRFV-1-F | AGATTTCCCTG | SEQ ID NO: 44 |
| | GCTTTTGGA | |
| LM_TBRFV-1-R | CTCTTTCTGAT | SEQ ID NO: 45 |
| | ATCAAGCACT | |
| LM_TBRFV-1- | CAAGGAGAGAC | SEQ ID NO: 46 |
| probe | TGCTAAATCGG | |

The amplified fragment is 187 bp.

### Mix:

Kit TaqMan universal Master Mix, Applied Biosystem ThermoFisher Scientific.

| Product | Initial concentration | Final concentration | μL/well |
|---|---|---|---|
| Water (nuclease free) | | Qsp 20 μL | 7.1 |
| Primer Reverse LM_TBRFV-1-R | 10 μM | 0.5 μM (200 nM-1 μM) | 1 |
| Primer Forward LM_TBRFV-1-F | 10 μM | 0.5 μM (200 nM-1 μM) | 1 |
| Probe LM_TBRFV-1-probe | 5 μM | 0.25 μM (100-300 nM) | 0.5 |
| Gotaq Probe qPCR master mix | 2X | 1X | 10 |
| GoScript RT Mix for 1 step RT-qPCR | 50X | 1X | 0.4 |

The total volume of the mix is 20 μL; 2 μL of RNA are added; such that the final volume is 22 μL.

### Thermocycler:

Reverse transcription: 15 minutes at 45° C.;
Inactivation of Reverse transcription and Activation: 2 minutes at 95° C.;
cycles comprising:
Denaturation: 15 seconds at 95° C.,
Annealing primers: 15 seconds at 54° C.; and
Annealing probes: 30 seconds at 48° C.

For each sample, 3 replicates are performed. Standard dilution curves are used for the relative quantification.

A melting curve is performed at the end of the protocol in the StepOne® to ensure the specificity of the detection/quantification

The Ct of each sample is reported on the standard curve to calculate the relative quantity of virus in each sample.

### Results:

Anabelle tomato line, which is susceptible to ToMV, TMV, ToBRFV and ToMMV is used for Agrobacterium transformation with Tm2-2 variants as identified in the previous sections of the results. Infection by ToBRFV was carried out as disclosed on point 1.4, on transformants (presence of the T-DNA is checked as disclosed above) and on different controls (untransformed Anabelle). The phenotype of the plants is then scored at 14 DPI and 21 DPI. Presence of viral DNA is quantified by qPCR at 28 DPI, giving rise to a Ct value. The Ct or threshold cycle value is the cycle number at which the fluorescence generated within a reaction crosses the fluorescence threshold, corresponding to a fluorescent signal significantly above the background fluorescence. At the threshold cycle (Ct), a detectable amount of the amplified product has been generated during the early exponential phase of the reaction. The threshold cycle is inversely proportional to the original relative expression level of the gene of interest, i.e. the higher the Ct value, the higher the resistance level (it means the virus multiplication in plant is lower). The value of the susceptible plants transformed by a T-DNA providing an unmutated sequence of Tm2-2 can be used as control. A difference of Ct of 3.32 means a difference of 10 fold regarding virus quantity in the sample.

The test is reproduced twice (test 1 and test 2).

The results are detailed in the following table 13.

In this table, REP means the repetition number, “Plt nb” means the number of the plant. When “present” is indicated in the column “T-DNA genotype”, this means that the presence of the T-DNA has been checked by PCR, as disclosed above.

The results reported in this table clearly show that the resistance observed in the surrogate assay with transient expression in N. benthamiana is indeed representative of the resistance in tomato plants comprising the resistance gene.

These results moreover demonstrate that the mutant TM-2-2 gene, with at least the C848R mutation provides resistance against ToBRFV infection, and that the presence of the F655L mutation may improve the resistance.

The results regarding the resistance evaluated by visual symptoms (AUDPC Area under the disease progress curve at 0, 14 and 21 DPI) and the resistance evaluated by qPCR Ct (corresponding to viral presence) can be summarized in the following table depending on the genotype. The visual symptoms are evaluated with respect to a susceptible control, wherein “+” means less symptoms than the control, and “−” means no improvement. The Ct evaluated by qPCR is also evaluated with respect to a susceptible control, wherein “+” means less viral sequences detected and “−” means no improvement.

| genotype | description | Visual symptoms | qPCR Ct |
|---|---|---|---|
| TM-2-2 | Negative control | Control | control |
| | (transformed) | | |
| TM-2-2 with mutation | Single mutation | + | + |
| C848R | | | |
| TM-2-2 with mutations | Double mutation | + | ++ |
| C848R and F665L | | | |
| Anabelle | Susceptible control | − | − |
| | (untransformed) | | |
| Resistance source | Resistant control | + | Not tested |
| | (untransformed | | |

The inventors have then checked the resistance to other tobamoviruses, especially ToMV race 0, TMV and ToMMV of the plants transformed with T-DNA comprising the Tm2-2 variant with the double mutation (C848R and F655L). The results are reported in table 14 and clearly show that the plants comprising the T-DNA (i.e. marked as “present” in the last column) are resistant to all these tobamoviruses, in addition to ToBRFV as shown in table 13 (same mutant code).

These results entirely confirm the results presented in the preceding examples, namely that mutants of the TM-2-2 gene may provide resistance against ToBRFV infection to tomato plants, whilst simultaneously providing resistance against ToMV, TMV and ToMMV.

TABLE 13
results of ToBRFV infection of initially susceptible plants, transformed with different T-DNA, or untransformed.

| Test | | | | Plt | T-DNA | SCORING | SCORING | ct 28 |
|---|---|---|---|---|---|---|---|---|
| nb | DESCRIPTION | Mutant plant code | REP | Nb | GENOTYPE | 14 DPI | 21 DPI | DPI |
| 1 | Susceptible variety transformed with Tm2-14-25 | 52T-SL-ANA-A1224-1099-3745 | 1 | 9 | PRESENT | 9 | 7 | 24.19 |
| 1 | Susceptible variety transformed with Tm2-14-25 | 52T-SL-ANA-A1224-1099-3745 | 3 | 7 | PRESENT | 7 | 7 | 21.94 |
| 1 | Susceptible variety transformed with Tm2-467 | 7T-SL-ANA-A1225-990-3331 | 1 | 7 | PRESENT | 9 | 7 | 14.54 |
| 1 | Susceptible variety transformed with Tm2-467 | 7T-SL-ANA-A1225-990-3331 | 2 | 5 | PRESENT | 9 | 7 | 14.09 |
| 1 | Susceptible variety transformed with Tm2-467 | 7T-SL-ANA-A1225-983-3421 | 3 | 9 | PRESENT | 7 | 7 | 7.58 |
| 1 | Susceptible variety without Tm2² | na | 1 | 1 | | 5 | 1 | |
| 1 | Susceptible variety without Tm2² | na | 1 | 2 | | 5 | 1 | |
| 1 | Susceptible variety without Tm2² | na | 1 | 3 | | 5 | 1 | |
| 1 | Susceptible variety without Tm2² | na | 1 | 4 | | 3 | 1 | |
| 1 | Susceptible variety without Tm2² | na | 1 | 5 | | 3 | 1 | |
| 1 | Susceptible variety without Tm2² | na | 1 | 6 | | 5 | 1 | |
| 1 | Susceptible variety without Tm2² | na | 1 | 7 | | 5 | 3 | |
| 1 | Susceptible variety without Tm2² | na | 1 | 8 | | 5 | 1 | |
| 1 | Susceptible variety with Tm2² (without mutation) | 54T-SL-ANA-A1250-1074-3521 | 1 | 1 | PRESENT | 5 | 1 | |
| 1 | Susceptible variety with Tm2² (without mutation) | 54T-SL-ANA-A1250-1074-3521 | 1 | 4 | PRESENT | 5 | 3 | |
| 1 | Susceptible variety with Tm2² (without mutation) | 54T-SL-ANA-A1250-1074-3521 | 1 | 6 | PRESENT | 5 | 1 | 10.73 |
| 1 | Susceptible variety with Tm2² (without mutation) | 54T-SL-ANA-A1250-1074-3521 | 1 | 8 | PRESENT | 7 | 3 | |
| 1 | Susceptible variety with Tm2² (without mutation) | 54T-SL-ANA-A1250-1074-3521 | 1 | 9 | PRESENT | 5 | 3 | |
| 1 | Susceptible variety with Tm2² (without mutation) | 54T-SL-ANA-A1250-1074-3521 | 2 | 1 | PRESENT | 5 | 5 | |
| 1 | Susceptible variety with Tm2² (without mutation) | 54T-SL-ANA-A1250-1074-3521 | 2 | 2 | PRESENT | 7 | 1 | |
| 1 | Susceptible variety with Tm2² (without mutation) | 54T-SL-ANA-A1250-1074-3521 | 2 | 4 | PRESENT | 9 | 3 | |
| 1 | Susceptible variety with Tm2² (without mutation) | 54T-SL-ANA-A1250-1074-3521 | 2 | 6 | PRESENT | 5 | 3 | |
| 1 | Susceptible variety with Tm2² (without mutation) | 54T-SL-ANA-A1250-1074-3521 | 2 | 7 | PRESENT | 7 | 1 | 10.83 |
| 1 | Susceptible variety with Tm2² (without mutation) | 54T-SL-ANA-A1250-1074-3521 | 2 | 9 | PRESENT | 5 | 1 | |
| 1 | Susceptible variety with Tm2² (without mutation) | 54T-SL-ANA-A1250-1074-3521 | 3 | 3 | PRESENT | 5 | 3 | |
| 1 | Susceptible variety with Tm2² (without mutation) | 54T-SL-ANA-A1250-1074-3521 | 3 | 5 | PRESENT | 5 | 5 | 11.05 |
| 1 | Susceptible variety with Tm2² (without mutation) | 54T-SL-ANA-A1250-1074-3521 | 3 | 6 | PRESENT | 7 | 3 | |
| 1 | Susceptible variety with Tm2² (without mutation) | 54T-SL-ANA-A1250-1074-3521 | 3 | 7 | PRESENT | 5 | 5 | 7.06 |
| 1 | Susceptible variety with Tm2² (without mutation) | 54T-SL-ANA-A1250-1074-3521 | 3 | 8 | PRESENT | 5 | 3 | |
| 2 | Susceptible variety without Tm2² | | 1 | P2 | | 3 | 3 | |
| 2 | Susceptible variety without Tm2² | | 1 | P3 | | 3 | 3 | |
| 2 | Susceptible variety without Tm2² | | 1 | P5 | | 3 | 3 | |
| 2 | Susceptible variety without Tm2² | | 1 | P6 | | 3 | 3 | |
| 2 | Susceptible variety without Tm2² | | 1 | P7 | | 3 | 3 | |
| 2 | Susceptible variety without Tm2² | | 1 | P8 | | 3 | 3 | |
| 2 | Susceptible variety transformed with Tm2-14-25 | 52T-SL-ANA-A1224-1099-3745 | 2 | P14 | PRESENT | 9 | 7 | 21.16 |
| 2 | Susceptible variety transformed with Tm2-14-25 | 52T-SL-ANA-A1224-1099-3745 | 2 | P15 | PRESENT | 7 | 7 | 26.83 |
| 2 | Susceptible variety transformed with Tm2-14-25 | 52T-SL-ANA-A1224-1099-3582 | 2 | P3 | PRESENT | 9 | 7 | 25.74 |
| 2 | Susceptible variety transformed with Tm2-14-25 | 52T-SL-ANA-A1224-1099-3582 | 2 | P9 | PRESENT | 9 | 7 | 27.57 |
| 2 | Susceptible variety transformed with Tm2-14-25 | | 1 | P2 | PRESENT | 5 | 7 | 22.35 |

TABLE 14
resistance of the transformants, transformed with Tm2-14-25 (double mutants) to ToMV,
TMV and ToMMV. R stands for ″resistant″ and S for ″susceptible″.

| Mutant plant code | Plant Nb | Strain | Score | T-DNA |
|---|---|---|---|---|
| 52T-SL-ANA-A1224-1099-3745 | 2 | ToMV race0 | S | |
| 52T-SL-ANA-A1224-1099-3745 | 3 | ToMV race0 | S | |
| 52T-SL-ANA-A1224-1099-3745 | 4 | ToMV race0 | S | |
| 52T-SL-ANA-A1224-1099-3745 | 5 | ToMV race0 | S | |
| 52T-SL-ANA-A1224-1099-3745 | 7 | ToMV race0 | S | |
| 52T-SL-ANA-A1224-1099-3745 | 9 | ToMV race0 | S | |
| 52T-SL-ANA-A1224-1099-3745 | 10 | ToMV race0 | S | |
| 52T-SL-ANA-A1224-1099-3745 | 12 | ToMV race0 | S | |
| 52T-SL-ANA-A1224-1099-3745 | 14 | ToMV race0 | R | Present |
| 52T-SL-ANA-A1224-1099-3745 | 15 | ToMV race0 | S | |
| 52T-SL-ANA-A1224-1099-3745 | 17 | ToMV race0 | R | Present |
| 52T-SL-ANA-A1224-1099-3745 | 19 | ToMV race0 | S | |
| 52T-SL-ANA-A1224-1099-3745 | 20 | ToMV race0 | S | |
| 52T-SL-ANA-A1224-1099-3745 | 25 | ToMV race0 | R | Present |
| 52T-SL-ANA-A1224-1099-3745 | 28 | ToMV race0 | S | |
| 52T-SL-ANA-A1224-1099-3745 | 29 | ToMV race0 | S | |
| 52T-SL-ANA-A1224-1099-3745 | 30 | ToMV race0 | R | Present |
| 52T-SL-ANA-A1224-1099-3745 | 31 | ToMV race0 | S | |
| 52T-SL-ANA-A1224-1099-3745 | 32 | ToMV race0 | S | |
| 52T-SL-ANA-A1224-1099-3745 | 33 | ToMV race0 | S | |
| 52T-SL-ANA-A1224-1099-3745 | 34 | ToMV race0 | S | |
| 52T-SL-ANA-A1224-1099-3745 | 36 | ToMV race0 | S | |
| 52T-SL-ANA-A1224-1099-3745 | 39 | ToMV race0 | R | Present |
| 52T-SL-ANA-A1224-1099-3745 | 41 | ToMV race0 | S | |
| 52T-SL-ANA-A1224-1099-3745 | 42 | ToMV race0 | S | |
| 52T-SL-ANA-A1224-1099-3745 | 44 | ToMV race0 | S | |
| 52T-SL-ANA-A1224-1099-3745 | 47 | ToMV race0 | S | |
| 52T-SL-ANA-A1224-1099-3745 | 49 | ToMV race0 | S | |
| 52T-SL-ANA-A1224-1099-3745 | 51 | ToMV race0 | S | |
| 52T-SL-ANA-A1224-1099-3745 | 53 | ToMV race0 | S | |
| 52T-SL-ANA-A1224-1099-3745 | 54 | ToMV race0 | S | |
| 52T-SL-ANA-A1224-1099-3745 | 3 | TMV | S | |
| 52T-SL-ANA-A1224-1099-3745 | 5 | TMV | S | |
| 52T-SL-ANA-A1224-1099-3745 | 6 | TMV | S | |
| 52T-SL-ANA-A1224-1099-3745 | 8 | TMV | S | |
| 52T-SL-ANA-A1224-1099-3745 | 9 | TMV | S | |
| 52T-SL-ANA-A1224-1099-3745 | 14 | TMV | R | Present |
| 52T-SL-ANA-A1224-1099-3745 | 15 | TMV | S | |
| 52T-SL-ANA-A1224-1099-3745 | 16 | TMV | R | Present |
| 52T-SL-ANA-A1224-1099-3745 | 18 | TMV | S | |
| 52T-SL-ANA-A1224-1099-3745 | 19 | TMV | S | |
| 52T-SL-ANA-A1224-1099-3745 | 20 | TMV | S | |
| 52T-SL-ANA-A1224-1099-3745 | 21 | TMV | S | |
| 52T-SL-ANA-A1224-1099-3745 | 22 | TMV | S | |
| 52T-SL-ANA-A1224-1099-3745 | 23 | TMV | S | |
| 52T-SL-ANA-A1224-1099-3745 | 24 | TMV | R | Present |
| 52T-SL-ANA-A1224-1099-3745 | 25 | TMV | S | |
| 52T-SL-ANA-A1224-1099-3745 | 28 | TMV | S | |
| 52T-SL-ANA-A1224-1099-3745 | 32 | TMV | S | |
| 52T-SL-ANA-A1224-1099-3745 | 34 | TMV | R | Present |
| 52T-SL-ANA-A1224-1099-3745 | 36 | TMV | S | |
| 52T-SL-ANA-A1224-1099-3745 | 38 | TMV | S | |
| 52T-SL-ANA-A1224-1099-3745 | 39 | TMV | S | |
| 52T-SL-ANA-A1224-1099-3745 | 40 | TMV | R | Present |
| 52T-SL-ANA-A1224-1099-3745 | 42 | TMV | R | Present |
| 52T-SL-ANA-A1224-1099-3745 | 46 | TMV | R | Present |
| 52T-SL-ANA-A1224-1099-3745 | 47 | TMV | S | |
| 52T-SL-ANA-A1224-1099-3745 | 51 | TMV | S | |
| 52T-SL-ANA-A1224-1099-3745 | 52 | TMV | R | Present |
| 52T-SL-ANA-A1224-1099-3745 | 53 | TMV | S | |
| 52T-SL-ANA-A1224-1099-3745 | 54 | TMV | R | Present |
| 52T-SL-ANA-A1224-1099-3745 | 2 | ToMMV | R | Present |
| 52T-SL-ANA-A1224-1099-3745 | 3 | ToMMV | R | Present |
| 52T-SL-ANA-A1224-1099-3745 | 4 | ToMMV | S | |
| 52T-SL-ANA-A1224-1099-3745 | 5 | ToMMV | S | |
| 52T-SL-ANA-A1224-1099-3745 | 6 | ToMMV | S | |
| 52T-SL-ANA-A1224-1099-3745 | 8 | ToMMV | R | Present |
| 52T-SL-ANA-A1224-1099-3745 | 10 | ToMMV | R | Present |
| 52T-SL-ANA-A1224-1099-3745 | 11 | ToMMV | S | |
| 52T-SL-ANA-A1224-1099-3745 | 12 | ToMMV | S | |
| 52T-SL-ANA-A1224-1099-3745 | 13 | ToMMV | S | |
| 52T-SL-ANA-A1224-1099-3745 | 14 | ToMMV | R | Present |
| 52T-SL-ANA-A1224-1099-3745 | 15 | ToMMV | S | |
| 52T-SL-ANA-A1224-1099-3745 | 17 | ToMMV | R | Present |
| 52T-SL-ANA-A1224-1099-3745 | 18 | ToMMV | S | |
| 52T-SL-ANA-A1224-1099-3745 | 19 | ToMMV | S | |
| 52T-SL-ANA-A1224-1099-3745 | 20 | ToMMV | S | |
| 52T-SL-ANA-A1224-1099-3745 | 21 | ToMMV | S | |
| 52T-SL-ANA-A1224-1099-3745 | 23 | ToMMV | S | |
| 52T-SL-ANA-A1224-1099-3745 | 24 | ToMMV | S | |
| 52T-SL-ANA-A1224-1099-3745 | 26 | ToMMV | S | |
| 52T-SL-ANA-A1224-1099-3745 | 29 | ToMMV | S | |
| 52T-SL-ANA-A1224-1099-3745 | 30 | ToMMV | S | |
| 52T-SL-ANA-A1224-1099-3745 | 31 | ToMMV | S | |
| 52T-SL-ANA-A1224-1099-3745 | 32 | ToMMV | S | |
| 52T-SL-ANA-A1224-1099-3745 | 34 | ToMMV | S | |
| 52T-SL-ANA-A1224-1099-3745 | 36 | ToMMV | S | |
| 52T-SL-ANA-A1224-1099-3745 | 37 | ToMMV | R | Present |
| 52T-SL-ANA-A1224-1099-3745 | 39 | ToMMV | S | |
| 52T-SL-ANA-A1224-1099-3745 | 40 | ToMMV | S | |
| 52T-SL-ANA-A1224-1099-3745 | 41 | ToMMV | S | |
| 52T-SL-ANA-A1224-1099-3745 | 42 | ToMMV | S | |
| 52T-SL-ANA-A1224-1099-3745 | 44 | ToMMV | S | |
| 52T-SL-ANA-A1224-1099-3745 | 45 | ToMMV | S | |
| 52T-SL-ANA-A1224-1099-3745 | 49 | ToMMV | R | Present |
| 52T-SL-ANA-A1224-1099-3745 | 50 | ToMMV | S | |
| 52T-SL-ANA-A1224-1099-3745 | 52 | ToMMV | S | |
| 52T-SL-ANA-A1224-1099-3745 | 53 | ToMMV | S | |

### BIBLIOGRAPHY

Baggs, E., Dagdas, G., and Krasileva, K. V. 2017. NLR diversity, helpers and integrated domains: making sense of the NLR IDentity. Curr Opin Plant Biol 38:59-67.
Calder, V. L., and Palukaitis, P. 1992. Nucleotide sequence analysis of the movement genes of resistance breaking strains of tomato mosaic virus. J Gen Virol 73 (Pt 1):165-168.
Ishibashi et al, 2007. An inhibitor of viral RNA replication is encoded by a plant resistance gene. PNAS 104 (34) 13833-13838.
Kapos, P., Devendrakumar, K. T., and Li, X. 2019. Plant NLRs: From discovery to application. Plant Sci 279:3-18.
Kobayashi, M., Yamamoto-Katou, A., Katou, S., Hirai, K., Meshi, T., Ohashi, Y., and Mitsuhara, I. 2011. Identification of an amino acid residue required for differential recognition of a viral movement protein by the Tomato mosaic virus resistance gene Tm-2(2). J Plant Physiol 168:1142-1145.
Lanfermeijer, F. C., Warmink, J., and Hille, J. 2005. The products of the broken Tm-2 and the durable Tm-2(2) resistance genes from tomato differ in four amino acids. J Exp Bot 56:2925-2933.
Lanfermeijer, F. C., Dijkhuis, J., Sturre, M. J., de Haan, P., and Hille, J. 2003. Cloning and characterization of the durable tomato mosaic virus resistance gene Tm-2(2) from Lycopersicon esculentum. Plant Mol Biol 52:1037-1049.
Lanfermeirjer, F., Jiang, G, Ferwerda, M A, Kijkhuis, J, de Haan, P, Yang, R, Hille, J. 2004. The durable resistance gene Tm-22 from tomato confers resistance against ToMV in tobacco and preserves its viral specificity. Plant Science 167:687-692.
Luria N. et al. 2017. A New Israeli Tobamovirus Isolate Infects Tomato Plants Harboring Tm-2² Resistance Genes. PLoS One; 12(1): e0170429.
Ma, L., Lukasik, E, Gawehns F, Takken F L W. 2012. The use of agroinfiltration for transient expression of plant resistance and fungal effector proteins in Nicotiana benthamiana leaves. Methods Mol Biol 835:61-74.
Meshi, T., Motoyoshi, F., Maeda, T., Yoshiwoka, S., Watanabe, H., and Okada, Y. 1989. Mutations in the tobacco mosaic virus 30-kD protein gene overcome Tm-2 resistance in tomato. Plant Cell 1:515-522.
Mondragon-Palomino, M., Meyers, B. C., Michelmore, R. W., and Gaut, B. S. 2002. Patterns of positive selection in the complete NBS-LRR gene family of Arabidopsis thaliana. Genome Res 12:1305-1315.
Nagai, A., Duarte M. L. L., Chaves A. LR., Peres L. EP., dos Santos D. Y. A. C. 2019. Tomato mottle mosaic virus in Brazil and its relationaship with Tm-2² gene. Eur J Plant Pathol 155, 353-359.
Salem N. et al, 2015. A new tobamovirus infecting tomato crops in Jordan. Arch. Virol. 161 (2), 503-506.
Segretin, M. E., Pais, M., Franceschetti, M., Chaparro-Garcia, A., Bos, J. I. B., Banfield, M. J., and Kamoun, S. 2014. Single Amino Acid Mutations in the Potato Immune Receptor R3a Expand Response to Phytophthora Effectors. Molecular Plant-Microbe Interactions@ 27:624-637.
Slootweg, E., Koropacka, K., Roosien, J., Dees, R., Overmars, H., Lankhorst, R. K., van Schaik, C., Pomp, R., Bouwman, L., Helder, J., Schots, A., Bakker, J., Smant, G., and Goverse, A. 2017. Sequence Exchange between Homologous NB-LRR Genes Converts Virus Resistance into Nematode Resistance, and Vice Versa. Plant Physiol 175:498-510.
Sui, X. et al, 2017. Molecular and Biological Characterization of Tomato mottle mosaic virus and Development of RT-PCR Detection. Plant Disease 101, 704-711.
Tomita, R., Sekine, K. T., Tateda, C., and Kobayashi, K. 2019. Identification and Functional Analysis of NB-LRR-Type Virus Resistance Genes: Overview and Functional Analysis of Candidate Genes. Methods Mol Biol 2028:1-10.
Wang, J., Chen, T., Han, M., Qian, L., Li, J., Wu, M., Han, T., Cao, J., Nagalakshmi, U., Rathjen, J. P., Hong, Y., and Liu, Y. 2020. Plant NLR immune receptor Tm-22 activation requires NB-ARC domain-mediated self-association of CC domain. PLoS Pathog 16:e1008475.
Weber, H., and Pfitzner, A. J. 1998. Tm-2(2) resistance in tomato requires recognition of the carboxy terminus of the movement protein of tomato mosaic virus. Mol Plant Microbe Interact 11:498-503.
Weber, H., Schultze, S., and Pfitzner, A. J. 1993. Two amino acid substitutions in the tomato mosaic virus 30-kilodalton movement protein confer the ability to overcome the Tm-2(2) resistance gene in the tomato. J Virol 67:6432-6438.
Weber, H., Ohnesorge, S., Silber, M. V., and Pfitzner, A. J. 2004. The Tomato mosaic virus 30 kDa movement protein interacts differentially with the resistance genes Tm-2 and Tm-2(2). Arch Virol 149:1499-1514.

1-29. (canceled)

30. A TM-2-2 protein variant, which has at least 90% amino acid sequence identity with SEQ ID No:8,
wherein said TM-2-2 protein variant confers resistance against at least ToBRFV infection in tomatoes, and
wherein said TM-2-2 protein variant comprises
a tyrosine (Y), a phenylalanine (F) or a tryptophan (W) at the position corresponding to tyrosine 767 of SEQ ID No:8, and
one or more of the following substitutions:
an arginine (R) at the position corresponding to cysteine 848 of the TM-2-2 protein (SEQ ID No:8);
a cysteine (C), a phenylalanine (F), a methionine (M), a tyrosine (Y) or a tryptophan (W) at the position corresponding to asparagine 822 of the TM-2-2 protein (SEQ ID No:8), and
a histidine (H), a lysine (K) or a threonine (T) at the position corresponding to serine 825 of the TM-2-2 protein (SEQ ID No:8).

31. The TM-2-2 protein variant according to claim 30, further comprising a leucine at the position corresponding to phenylalanine 655 of the TM-2-2 protein (SEQ ID NO:8).

32. A resistance gene encoding the TM-2-2 protein variant according to claim 30, conferring resistance to ToBRFV infection in tomato plants.

33. A nucleic acid construct comprising a sequence encoding the TM-2-2 protein variant according to claim 30.

34. A plant cell, comprising the resistance gene according to claim 32 within its genomic DNA, homozygously or heterozygously.

35. The cell according to claim 34 or a tissue culture of said cells, wherein the cells are derived from embryos, protoplasts, meristematic cells, callus, pollen, leaves, anthers, stems, petioles, roots, root tips, seeds, flowers, cotyledons, and/or hypocotyls, and contain in their genome said resistance gene.

36. A plant resistant against ToBRFV,
wherein said plant comprises in its genome the resistance gene according to claim 32, homozygously or heterozygously.

37. A S. lycopersicum plant resistant to ToBRFV, comprising a mutated Tm-2-2 gene encoding a variant of the TM-2-2 protein (SEQ ID No:8),
wherein said TM-2-2 protein variant has at least 90% sequence identity with SEQ ID No:8,
wherein the tyrosine (Y) at the position corresponding to position 767 of SEQ ID No:8 is not mutated or is substituted by a phenylalanine (F) or a tryptophan (W) and
wherein said TM-2-2 protein variant comprises one or more of the following substitutions:
the cysteine (C) at the position corresponding to position 848 of SEQ ID No:8 is substituted by an arginine (R),
the asparagine (N) at the position corresponding to position 822 of SEQ ID No:8 is substituted by a cysteine (C), a phenylalanine (F), a methionine (M), a tyrosine (Y) or a tryptophan (W), and
the serine (S) at the position corresponding to position 825 of SEQ ID No:8 is substituted by a histidine (H), a lysine (K) or a threonine (T).

38. The S. lycopersicum plant according to claim 37, wherein said mutated Tm-2-2 gene encodes a variant of the TM-2-2 protein further comprising a leucine at the position corresponding to phenylalanine 655 of the TM-2-2 protein (SEQ ID NO:8).

39. The S. lycopersicum plant according to claim 37, wherein said mutated Tm-2-2 gene encodes a variant of TM-2-2 protein, having SEQ ID No:9, SEQ ID No:10, SEQ ID No:17, SEQ ID No:18, SEQ ID No:19, SEQ ID No:20, SEQ ID No:21, SEQ ID No:22, SEQ ID No:23, SEQ ID No:24, SEQ ID No:25 or SEQ ID No:26.

40. The S. lycopersicum plant according to claim 37, wherein said mutated Tm-2-2 gene is obtained by gene editing, base-editing or prime-editing techniques.

41. A plant part of the S. lycopersicum plant resistant to ToBRFV, comprising a mutated Tm-2-2 gene encoding the variant of the TM-2-2 protein (SEQ ID No:8),
wherein said TM-2-2 protein variant has at least 90% sequence identity with SEQ ID No:8,
wherein the tyrosine (Y) at the position corresponding to position 767 of SEQ ID No:8 is not mutated or is substituted by the phenylalanine (F) or the tryptophan (W) and
wherein said TM-2-2 protein variant comprises one or more of the following substitutions:
the cysteine (C) at the position corresponding to position 848 of SEQ ID No:8 is substituted by an arginine (R),
the asparagine (N) at the position corresponding to position 822 of SEQ ID No:8 is substituted by the cysteine (C), the phenylalanine (F), the methionine (M), the tyrosine (Y) or the tryptophan (W), and
the serine (S) at the position corresponding to position 825 of SEQ ID No:8 is substituted by the histidine (H), the lysine (K) or the threonine (T), wherein said plant part comprises at least one cell according to claim 34.

42. A S. lycopersicum seed, which comprises at least one cell according to claim 34.

43. A method for obtaining transgenic S. lycopersicum plants resistant to ToBRFV, comprising:
obtaining a construct comprising a sequence encoding the TM-2-2 protein variant according to claim 30,
introducing said construct into a S. lycopersicum cell,
regenerating a transgenic plant;
optionally propagating the obtained plant.

44. A method for breeding a S. lycopersicum plant resistant to ToBRFV, and optionally to at least one of TMV, ToMV, and/or ToMMV, comprising:
crossing a S. lycopersicum plant comprising the resistance gene according to claim 32 with an initial S. lycopersicum plant devoid of resistance gene,
selecting in the progeny thus obtained, a plant bearing the resistance gene,
optionally self-pollinating one or several times the plant obtained at step (b) and selecting in the progeny thus obtained a plant bearing the resistance gene.

45. A method of producing a S. lycopersicum plant resistant to ToBRFV, and optionally to at least one of TMV, ToMV, and/or ToMMV, comprising:
obtaining a part of the plant according claim 37,
vegetatively propagating said plant part to generate a plant from said plant part.

46. A method for improving the yield of tomato plants or for reducing the loss on tomato production in an environment infested or likely to be infested by ToBRFV comprising growing tomato plants comprising in their genome the resistance gene according to claim 32.

47. A method for reducing the loss on tomato production in condition of ToMV, TMV, ToMMV and/or ToBRFV infestation, comprising growing a tomato plant comprising in its genome the resistance gene according to claim 32.

48. A method of producing tomatoes comprising:
growing the S. lycopersicum plant according to claim 37;
allowing said plant to set fruit; and
harvesting fruit of said plant.

49. A method for identifying, detecting and/or selecting mutants of the Tm-2-2 gene conferring resistance against ToBRFV, comprising:
expressing transiently or constitutively in a Solanaceae plant, a mutant of the Tm-2-2 gene to be tested in presence of the movement protein (MP) of ToBRFV,
detecting an interaction between the protein expressed from the mutant gene and the MP protein.


SEQUENCE LISTING (46 sequences)

SEQ ID NO: 1 - DNA, 2820 bp, Lycopersicon esculentum
atagaaaaaagaaatttctcctttttcattaatgtgcagctgccccacgttgtcactccccttcttctttataatttccttcttgacaattattaggagacttggccgaggactccatctaccactaaaaagctaaagccatcagtatactcattttttggtagctactgaaaaagagagaaaaaaaaatggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacgatccaataagttcaattattgccttaagacgagttcttttgcggatgagtttgctatggagattgagaagataaagagaagggttgttgacattgaccgaataaggaaaacttacaacatcatagatacagataacaataatgatgattgtgttttgctggatcggagaagattattcctacatgctgatgaaacagagatcatcggtttggatgatgacttcaatatgctacaagccaaattactcaatcaagatttgcattatggagttgtttccatagttggcatgcccggtctggggaaaacaactcttgccaagaaactttataggctcattcgtgatcaatttgagtgttctggactggtctacgtttcacaacagccaagagcgggtgaaatcttacttgacattgccaaacaaattggactgacggaacagaaaattaaggaaaatttggaggacaacctgcgatcactcttgaaaataaaaaggtatgttatcctcctagatgacatttgggatgttgaaatttgggatgatctgaaacttgtccttcctgaatgtgactcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagctgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttaccgattgcctcaaggccatgtttcttgtactttagcctttaccccgaggaccatgaaattcgtgcttttgatttgataaatatgtggattgctgagaagtttattgtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgtatttggtgatcccggcaatgtcgctaggcttcgaaggattacattctactctgacaatgtcatgattgagttcttcggttctaatcctaagcttgagaagcttcgtgtacttttctgtttcacaaaagacccttccatattttctcatatggcttgttttgacttcaaattgttgcacacattggttgtagtcatgtctcaaagttttcaagcatatgtcactatcccaagcaaatttgggaacatgacttgcttacgctatctgaaattggaggggaatatttgtggaaaactgccaaatagtattgtcaagctcacacgtctagagaccatagacattgatcgacgtagcctcattcaacttccttctggtgtttgggagtctaaacatttgagacatctttgttatagagattatggacaagcatgtaacagttgcttttctataagctcattttacccaaacatttactcattgcatcctaacaatctacaaaccttgatgtggatacctgataaattttttgaaccgaggttgttgcaccgattgatcaatttaagaaaactgggtatactgggagtgtccaattcaaccgttaagatattatcaacatgtcgccctgtgccaaaggcgctaaaggttctgaagctcaggtttttcagtgatccgagtgagcaaataaacttgtcatcctatccaaaaattgttaagttgcatttgaatgttgacagaacaatagccttgaactctgaagcattccctccaaatattatcaagcttactcttgtctgctttatggtagacagttgtctactggcagtgcttaagacattacccaaattaagaaaacttaaaatggtcatctgcaagtataatgaagaaaagatggctctctcgggcgaggcaaatggttatagctttccgcaacttgaagttttgcatattcatagcccgaatgggttgtctgaagtaacatgcacggatgatgtcagtatgcccaaattgaaaaagctgttacttacaggattccattgcggaatcagtttatcggaacggcttaaaaagctgagtaaatgaacatctcaacaggtcagtttgctagtataactatttacgtacaggg

SEQ ID NO: 2 - DNA, 2819 bp, Lycopersicon esculentum
atagaaaaaagaaatttctcctttttcattaatgtgcagctgccccacgttgtcactccccttcttctttatcatttccttcttgacattattaggagacttggccgtggactccatctaccactaaaaagctaaagccatcagtatactcattttttggtagctactgaaaaagagagaaaaaaaaatggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcggatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttttcctcctagatgacatttgggatgtggaaatttgggatgatctgaaacttgtccttcctgaatgtgattcaaaagtcggcagtagaattataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgctcaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagccaactttacggtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatgaacatctcaacaggtcagtttgctagtataactatttacgtacaggg

SEQ ID NO: 3 - DNA, 2586 bp, Lycopersicon esculentum
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 4 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagaaaccatagacattgatcgacgtagcctcattcaacctccttctggtgtttgggagtctaaacatttgagacatctttgttatagagattatggacaagcatgtaacagttgcttttctataagctcattatacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccatcgccgaatcagtttatcggaacggcttaaaaagctgagtaaatga

SEQ ID NO: 5 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagaaaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccatcgccgaatcagtttatcggaacggcttaaaaagctgagtaaatga

SEQ ID NO: 6 - protein, 861 aa, Lycopersicon esculentum
MAEILLTSVINKSVEIAGNLLIQEGKRLYWLKEDIDWLQREMRHIRSYVDNAKAKEAGGDSRVKNLLKDIQELAGDVEDLLDDFLPKIQRSNKFNYCLKTSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRAGEILLDIAKQIGLTEQKIKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIAGRCGGIPLAIVVTAGMLRARERTEHAWNRVLESMGHKVQDGCAKVLALSYNDLPIASRPCFLYFSLYPEDHEIRAFDLINMWIAEKFIVVNSGNRREAEDLAEDVLNDLVSRNLIQLAKRTYNGRISSCRIHDLLHSLCVDLAKESNFFHTAHDVFGDPGNVARLRRITFYSDNVMIEFFGSNPKLEKLRVLFCFTKDPSIFSHMACFDFKLLHTLVVVMSQSFQAYVTIPSKFGNMTCLRYLKLEGNICGKLPNSIVKLTRLETIDIDRRSLIQLPSGVWESKHLRHLCYRDYGQACNSCFSISSFYPNIYSLHPNNLQTLMWIPDKFFEPRLLHRLINLRKLGILGVSNSTVKILSTCRPVPKALKVLKLRFFSDPSEQINLSSYPKIVKLHLNVDRTIALNSEAFPPNIIKLTLVCFMVDSCLLAVLKTLPKLRKLKMVICKYNEEKMALSGEANGYSFPQLEVLHIHSPNGLSEVTCTDDVSMPKLKKLLLTGFHCGISLSERLKKLSK

SEQ ID NO: 7 - protein, 861 aa, Lycopersicon esculentum
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVFLLDDIWDVEIWDDLKLVLPECDSKVGSRIIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLANFTVDRYILAVLKTFPKLRKLKMFICKYNEEKMDLSGEANGYSFPQLEVLHIHSPNGLSEVTCTDDVSMPKLKKLLLTGFHCRISLSERLKKLSK

SEQ ID NO: 8 - protein, 861 aa, Lycopersicon esculentum
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLSEVTCTDDVSMPKLKKLLLTGFHCRISLSERLKKLSK

SEQ ID NO: 9 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSLYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLSEVTCTDDVSMPKLKKLLLTGFHRRISLSERLKKLSK

SEQ ID NO: 10 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLSEVTCTDDVSMPKLKKLLLTGFHRRISLSERLKKLSK

SEQ ID NO: 11 - protein, 385 aa, Lycopersicon esculentum
CRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLSEVTCTDDVSMPKLKKLLLTGFHRRISLSERLKKLSK

SEQ ID NO: 12 - DNA, 1158 bp, Lycopersicon esculentum
tgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 13 - protein, 268 aa, Tobacco mosaic virus
MALVVKGKVNINEFIDLTKMEKILPSMFTPVKSVMCSKVDKIMVHENESLSGVNLLKGVKLIDSGYVCLAGLVVTGEWNLPDNCRGGVSVCLVDKRMERADEATLGSYYTAAAKKRFQFKVVPNYAITTQDAMKNVWQVLVNIRNVKMSAGFCPLSLEFVSVCIVYRNNIKLGLREKITNVRDGGPMELTEEVVDEFMEDVPMSIRLAKFRSRTGKKSDVRKGKNSSSDRSVPNKNYRNVKDFGGMSFKKNNLIDDDSEATVAESDSF

SEQ ID NO: 14 - protein, 264 aa, Tomato mosaic virus
MALVVKGKVNINEFIDLSKSEKLLPSMFTPVKSVMVSKVDKIMVHENESLSEVNLLKGVKLIEGGYVCLVGLVVSGEWNLPDNCRGGVSVCMVDKRMERADEATLGSYYTAAAKKRFQFKVVPNYGITTKDAEKNIWQVLVNIKNVKMSAGYCPLSLEFVSVCIVYKNNIKLGLREKVTSVNDGGPMELSEEVVDEFMENVPMSVRLAKFRTKSSKRGPKNNNNLGKGRSGGRSKPKSFDEVEKEFDNLIEDEAETSVADSDSY

SEQ ID NO: 15 - protein, 266 aa, Tomato brown rugose fruit virus
MALVKGKVNINEFIDLSKSEKFLPSMFTPVKSVMISKVDKILVHEDESLSEVNLLKGVKLIDGGYVHLAGLVVTGEWNLPDNCRGGVSVCLVDKRMERADEATLASYYTAAAKKRFQFKVVPNYNITTKDAEKAVWQVLVNIRNVKIAAGYCPLSLEFVSVCIVYKNIIKLGLREKITSVTDGGPMELSEEVVDEFMEEVPMSVRLAKFRSKTGKKFSSKSENNSGNNRPKPNKNQRKEKGLKVRVEKDNLIDNELETYVADSDSY

SEQ ID NO: 16 - protein, 268 aa, Tomato mottle virus
MALTVSGKVRISEFIDLSKSERLLPSMFTHVKSVSVSKVDKVMVNEEDSLSEVNLLKGVKLIDGGYVCLAGLVVSGEWNLPDNCRGGVSICLVDKRMQRADEATLGSYYTGAAKKRFQFKIVPNYAITTKDAEKNIWQVLVNIRNVKMAGGFCPLSLEFVSVCIVYKNNIKLGLREKITRVDDAGPIELTEEVVDEFMESVPMSVRLAKFRTKSSKRGPKHNSNNTNDRKGRSNFRKKQDQESYGVSDSLDNLIEDDTETSVAGSDSY

SEQ ID NO: 17 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAFFSVDRYILAVLKTFPKLRKLKMFICKYNEEKMDLSGEANGYSFPQLEVLHIHSPNGLSEVTCTDDVSMPKLKKLLLTGFHRRISLSERLKKLSK

SEQ ID NO: 18 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAWFSVDRYILAVLKTFPKLRKLKMFICKYNEEKMDLSGEANGYSFPQLEVLHIHSPNGLSEVTCTDDVSMPKLKKLLLTGFHRRISLSERLKKLSK

SEQ ID NO: 19 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLHEVTCTDDVSMPKLKKLLLTGFHCRISLSERLKKLSK

SEQ ID NO: 20 - protein, 861 aa, Lycopersicon esculentum
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLKEVTCTDDVSMPKLKKLLLTGFHCRISLSERLKKLSK

SEQ ID NO: 21 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPNGLTEVTCTDDVSMPKLKKLLLTGFHCRISLSERLKKLSK

SEQ ID NO: 22 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPCGLSEVTCTDDVSMPKLKKLLLTGFHCRISLSERLKKLSK

SEQ ID NO: 23 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPFGLSEVTCTDDVSMPKLKKLLLTGFHCRISLSERLKKLSK

SEQ ID NO: 24 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPMGLSEVTCTDDVSMPKLKKLLLTGFHCRISLSERLKKLSK

SEQ ID NO: 25 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPYGLSEVTCTDDVSMPKLKKLLLTGFHCRISLSERLKKLSK

SEQ ID NO: 26 - protein, 861 aa, artificial
  mutant
MAEILLTSVINKSVEIAGNLLIQEGKRLYWLKEDIDWLQREMRHIRSYVDNAKAKEAGGDSRVKNLLKDIQELAGDVEDLLDDFLPKIQQSNKFNYCLKRSSFADEFAMEIEKIKRRVVDIDRIRKTYNIIDTDNNNDDCVLLDRRRLFLHADETEIIGLDDDFNMLQAKLLNQDLHYGVVSIVGMPGLGKTTLAKKLYRLIRDQFECSGLVYVSQQPRASEILLDIAKQIGLTEQKMKENLEDNLRSLLKIKRYVILLDDIWDVEIWDDLKLVLPECDSKVGSRMIITSRNSNVGRYIGGESSLHALQPLESEKSFELFTKKIFNFDDNNSWANASPDLVNIGRNIVGRCGGIPLAIVVTAGMLRARERTEHAWNRVLESMGHKVQDGCAKVLALSYNDLPIASRPCFLYFGLYPEDHEIRAFDLINMWIAEKFIVVNSGNRREAEDLAEDVLNDLVSRNLIQLAKRTYNGRISSCRIHDLLHSLCVDLAKESNFFHTAHDAFGDPGNVARLRRITFYSDNVMIEFFRSNPKLEKLRVLFCFAKDPSIFSHMAYFDFKLLHTLVVVMSQSFQAYVTIPSKFGNMTCLRYLRLEGNICGKLPNSIVKLTRLETIDIDRRSLIQPPSGVWESKHLRHLCYRDYGQACNSCFSISSFYPNIYSLHPNNLQTLMWIPDKFFEPRLLHRLINLRKLGILGVSNSTVKMLSIFSPVLKALEVLKLSFSSDPSEQIKLSSYPHIAKLHLNVNRTMALNSQSFPPNLIKLTLAYFSVDRYILAVLKTFPKLRKLKMFICKYNEEKMDLSGEANGYSFPQLEVLHIHSPWGLSEVTCTDDVSMPKLKKLLLTGFHCRISLSERLKKLSK

SEQ ID NO: 27 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagccttttttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccatcgccgaatcagtttatcggaacggcttaaaaagctgagtaaatga

SEQ ID NO: 28 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctggtttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccatcgccgaatcagtttatcggaacggcttaaaaagctgagtaaatga

SEQ ID NO: 29 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgcatgaagtaacgtgcacggatgatgtcagtatgcccaaattgaaaaagctgttacttacaggattccattgccgaatcagtttatcggaacggcttaaaaagctgagtaaatga

SEQ ID NO: 30 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgaaggaagtaacgtgcacggatgatgtcagtatgcccaaattgaaaaagctgttacttacaggattccattgccgaatcagtttatcggaacggcttaaaaagctgagtaaatga

SEQ ID NO: 31 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgactgaagtaacgtgcacggatgatgtcagtatgcccaaattgaaaaagctgttacttacaggattccattgccgaatcagtttatcggaacggcttaaaaagctgagtaaatga

SEQ ID NO: 32 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgtgtgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 33 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgtttgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 34 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgatggggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 35 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgtatgggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 36 - DNA, 2586 bp, artificial
  mutant
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgtgggggttgtctgaagtaacgtgcacggatgatgtcagtatgcccaaattgaaaaagctgttacttacaggattccattgccgaatcagtttatcggaacggcttaaaaagctgagtaaatga

SEQ ID NO: 37 - DNA, 18 bp, artificial
  primer
cctgccgagaaagtatcc

SEQ ID NO: 38 - DNA, 18 bp, artificial
  primer
gccaacgctatgtcctga

SEQ ID NO: 39 - DNA, 21 bp, artificial
  primer
ttcctccaaatctcatcaagc

SEQ ID NO: 40 - DNA, 22 bp, artificial
  primer
caacaagccaagagaaaacaca

SEQ ID NO: 41 - DNA, 11668 bp, artificial
  plasmid
atggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagagaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccattgccgaatcagtttatcggaacggcttaaaaagctgagtaaatgaacctagggtgggatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataaggtgatatcgaattcctgcggtacccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtcactttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacaaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattatcagcttgcatgcagctctcccatatggtcgactagaggcctgccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgcaggtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacctcgagaattctcaacacaacatatacaaaacaaacgaatctcaagcaatcaagcattctacttctattgcagcaatttaaatcatttcttttaaagcaaaagcaattttctgaaaattttcaccatttacgaacgatagttaattaacc

SEQ ID NO: 42 - DNA, 11670 bp, artificial
  plasmid
acaaattgacgcttagacaacttaataacacattgcggacgtttttaatgtactgaattaacgccgaattgaattatcagcttgcatgcagctctcccatatggtcgactagaggcctgccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgcaggtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacctcgagaattctcaacacaacatatacaaaacaaacgaatctcaagcaatcaagcattctacttctattgcagcaatttaaatcatttcttttaaagcaaaagcaattttctgaaaattttcaccatttacgaacgatagttaattaaccatggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagaaaccatagacattgatcgacgtagcctcattcaacctccttctggtgtttgggagtctaaacatttgagacatctttgttatagagattatggacaagcatgtaacagttgcttttctataagctcattatacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccatcgccgaatcagtttatcggaacggcttaaaaagctgagtaaatgaagcttctagggtgggatatgaagatgaagatgaaatatttggtgtgtcaaataaaaagcttgtgtgcttaagtttgtgttttctcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataaggtgatatcgaattcctgcggtacccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtcactttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacaaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaa

SEQ ID NO: 43 - DNA, 11670 bp, artificial
  plasmid
acaaattgacgcttagacaacttaataacacattgcggacgtttttaatgtactgaattaacgccgaattgaattatcagcttgcatgcagctctcccatatggtcgactagaggcctgccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggactctagctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgcaggtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacctcgagaattctcaacacaacatatacaaaacaaacgaatctcaagcaatcaagcattctacttctattgcagcaatttaaatcatttcttttaaagcaaaagcaattttctgaaaattttcaccatttacgaacgatagttaattaaccatggctgaaattcttcttacatcagtaatcaataaatctgtagaaatagctggaaatttactgattcaagaaggaaagcgtttatattggttgaaagaggatatcgattggctccagagagaaatgagacacattcgatcttatgttgacaacgcaaaggccaaggaagctggaggtgattcaagggtcaaaaacttattgaaagatattcaagaattggcaggtgatgtggaggatctcttagatgacttccttccaaaaattcaacaatccaataagttcaattattgccttaagaggagttcttttgcagatgagtttgctatggagattgagaagataaagagaagggttgttgacattgaccgaataaggaaaacttacaacatcatagatacagataacaataatgatgattgtgttctgctggatcggagaagattattcctacatgctgatgaaacagagatcatcggtttggatgatgacttcaatatgctacaagccaaattacttaatcaagatttgcattatggagttgtttccatagttggcatgcccggtctggggaaaacaactcttgccaagaaactttataggctcattcgtgatcaatttgagtgttctggactggtctacgtttcacaacagccaagagcgagtgaaatcttacttgacattgccaaacaaattggactgacggaacagaaaatgaaggaaaatttggaggacaacctgcgatcactcttgaaaataaaaaggtatgttatcctcctagatgacatttgggatgtggaaatttgggatgatctgaaacttgtccttcctgaatgtgattcaaaagtcggcagtagaatgataatcacgtctcgaaatagtaatgtaggcagatacataggaggggaatcctccctccatgcattgcaacccctagaatccgagaaaagctttgaactctttaccaagaaaatctttaattttgatgataataatagttgggccaatgcttcacctgacttggtgaatattggtagaaatatagttgggagatgtggaggtataccgctagccatagtggtgactgcaggcatgttaagggcaagagaaagaacagaacatgcgtggaacagagtacttgagagtatgggccataaagttcaagatggatgtgctaaggtattggctctcagttacaatgatttacctattgcctcaaggccatgtttcttgtactttggcctttaccccgaggaccatgaaattcgtgcttttgatttgataaatatgtggattgctgagaagtttatagtagtaaatagtggtaataggcgagaggctgaggatttggcggaggacgtcctaaatgatttggtttctagaaacttgattcaacttgccaaaaggacatataatggaagaatttcaagttgtcgcatacatgacttgttacatagtttgtgtgtggacttggctaaggaaagtaacttctttcacaccgcgcatgatgcatttggtgatcccggcaatgttgctaggctccgaaggattacattctactctgacaatgtcatgattgagttcttccgttcaaatcctaagcttgagaagcttcgtgtacttttctgtttcgcaaaagacccttccatattttctcatatggcttattttgacttcaaattgttgcacacattggttgtagtcatgtctcaaagttttcaagcatatgtcactatcccaagcaaatttgggaacatgacttgcttacgctatctgagattggaggggaatatttgtggaaaactgccaaatagtattgtcaagctcacacgtctagaaaccatagacattgatcgacgtagcctcattcaacctccttctggtgtttgggagtctaaacatttgagacatctttgttatagagattatggacaagcatgtaacagttgcttttctataagctcattttacccaaatatttactcattgcatcctaacaatctacaaaccttgatgtggatacctgataaattttttgaaccgaggttgttgcaccgattgatcaatttaagaaaactgggtatactgggagtgtccaattctaccgttaagatgttatcaatatttagccctgtgcttaaggcgctggaggttctgaagctcagtttttccagtgacccgagtgaacaaataaagttgtcatcgtatccacatattgctaagttgcatttgaatgttaacagaacaatggccttgaactctcaatcatttcctccaaatctcatcaagcttactctagcctactttagtgtagaccgttatatactggcagtacttaagacatttcccaaattaagaaaacttaaaatgttcatctgcaagtataatgaagaaaagatggatctctcgggcgaggcaaatggttatagctttccgcaacttgaagttttgcatattcatagcccgaatgggttgtctgaagtaacgtgcacggatgatgtcagtatgcccaaattgaaaaagctgttacttacaggattccatcgccgaatcagtttatcggaacggcttaaaaagctgagtaaatgaagcttctagggtgggatatgaagatgaagatgaaatatttggtgtgtcaaataaaaagcttgtgtgcttaagtttgtgttttctcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataaggtgatatcgaattcctgcggtacccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtcactttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacaaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaa

SEQ ID NO: 44 - DNA, 20 bp, artificial
  primer
agatttccctggcttttgga

SEQ ID NO: 45 - DNA, 21 bp, artificial
  primer
ctctttctgatatcaagcact

SEQ ID NO: 46 - DNA, 22 bp, artificial
  probe
caaggagagactgctaaatcgg